(19) United States
(12) Patent Application Publication
(10) Pub. No.: US 2024/0002429 A1
(43) Pub. Date: Jan. 4, 2024

(54) PRODRUG OF 5a-HYDROXY-6ß-[2-(1H-IMIDAZOL-4-YL)ETHYLAMINO]CHOLESTAN-3ß-OL AND PHARMACEUTICAL COMPOSITIONS COMPRISING SAME FOR USE IN THE TREATMENT OF CANCER

(71) Applicant: DENDROGENIX, Liège (BE)

(72) Inventors: Stéphane SILVENTE, Liège (BE); Quentin MARLIER, Liège (BE); Arnaud RIVES, Liège (BE); Nicolas CARON, Liège (BE); Dario MOSCA, Liège (BE); Hélène MICHAUX, Liège (BE)

(73) Assignee: DENDROGENIX, Liège (BE)

(21) Appl. No.: 18/265,186

(22) PCT Filed: Dec. 3, 2021
(86) PCT No.: PCT/EP2021/084191

§ 371 Date: Jun. 2, 2023

(30) Foreign Application Priority Data
Dec. 3, 2020 (BE) 20205878

(51) Int. Cl. C07J 43/00; A61K 45/06; A61P 35/00

(52) U.S. Cl. CPC C07J 43/003; A61K 45/06; A61P 35/00

(57) ABSTRACT

The invention relates to a novel compound of general formula (I):

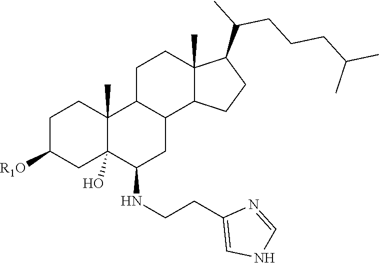

and/or a pharmaceutically acceptable salt of a compound of this kind, a pharmaceutical composition comprising at least said compound, for use thereof as a drug for causing regression of a mammalian cancerous tumor.

### FIELD OF THE INVENTION

The invention relates to the field of sterol compounds and more particularly to prodrugs of the compound 5α-hydroxy-6β-[2-(1H-imidazol-4-yl)-ethylamino]-cholestan-3β-ol and pharmaceutical compositions comprising same for use in particular in cancer treatment.

### BACKGROUND OF THE INVENTION

The term “cancer” or “cancerous tumor” covers a group of diseases characterized by the anarchic multiplication and propagation of abnormal cells. If the cancer cells are not eliminated, the progression of the disease will lead more or less quickly to the death of the person affected.

The management of cancer involves surgery, radiotherapy and chemotherapy, which may be used alone or in combination, simultaneously or sequentially. Chemotherapy uses antineoplastic agents, which are medicinal products that prevent or inhibit the maturation and proliferation of neoplasms. The antineoplastic agents act by effectively targeting the rapidly dividing cells. As the antineoplastic agents affect cell division, tumors with a high growth rate (such as acute myeloid leukemia and aggressive lymphomas, including Hodgkin disease) are more sensitive to chemotherapy, as a higher proportion of the cells targeted are undergoing cell division at any given moment. Malignant tumors with slower growth rates, such as indolent lymphomas, tend to respond much less markedly to chemotherapy. However, development of chemoresistance is a persistent problem during chemotherapy treatment. For example, the conventional treatment of acute myeloid leukemia (AML) comprises the combined administration of cytarabine with an anthracycline, such as daunorubicin. The overall survival rate at 5 years is 40% in young adults and about 10% in elderly patients. The response rates vary considerably with increasing age, from 40% to 55% in patients over 60 and from 24% to 33% in patients over 70. This is even worse for the elderly with unfavorable cytogenetic profiles and death in the 30 days following the treatment varies with age and severity from 10% to 50%. Moreover, restriction of the use of these molecules is also due to side effects, and in particular the emergence of chronic cardiac toxicity (linked to anthracyclines). The toxic mortality rate linked to intensive chemotherapy is from 10% to 20% in patients over 60.

With this risk-benefit profile of the conventional regime, only 30% of the elderly with recently diagnosed AML receive antineoplastic chemotherapy.

In recent decades, there has only been a slight improvement in the results for younger patients with AML, but none for adults over 60 (most of the patients with AML).

There is therefore a real need to develop molecules useful in the treatment of these cancerous tumors that have problems of chemoresistance and intrinsic toxicity of the antineoplastic drugs. The above data emphasize the need to find new approaches that combine both reducing the posology of antineoplastic agents for treating chemosensitive tumors and for evading the resistance of tumors resistant to antineoplastic drugs.

The compound 5α-hydroxy-6β-[2-(1H-imidazol-4-yl)-ethylamino]-cholestan-3β-ol is known from document EP3272350B1, known by the name Dendrogenin A, useful for treating drug-resistant tumors. Dendrogenin A (called DX101 hereinafter) is able to restore the sensitivity of drug-resistant tumors that are resistant to an antineoplastic agent or to increase the effects of the antineoplastic agents on tumors, which in its turn makes it possible to reduce the effective cytotoxic dose of antineoplastic agents against chemosensitive tumors.

The document of Medina et al. (J. Med. Chem., 2009, 52(23), 7765-77, XP009131948) describes compounds comprising, in position 3 of DX101, an acetate or butyrate radical, whose activities in vitro are different.

One aim of the present invention is to supply new compounds and prodrugs of the compound Dendrogenin A, useful in particular for treating cancerous tumors, chemosensitive and/or drug-resistant tumors.

The inventors have discovered, surprisingly, that specific C3 prodrugs of the compound Dendrogenin A display pharmacologic activity comparable to or greater than Dendrogenin A, in particular with good bioavailability as well as a prolonged therapeutic effect in the patient's body.

### SUMMARY OF THE INVENTION

The invention relates firstly to a compound of formula (I);

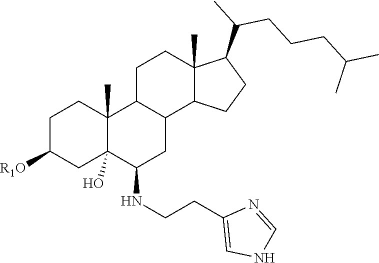

or a pharmaceutically acceptable salt of a compound of this kind, in which R₁ is selected from:
a group —C(O)NR₂R₃,
where R₂, R₃ are equivalent or different and are selected from H and a linear or branched, saturated or unsaturated C1 to C8 carbon chain optionally containing one or more substituents selected from allyl, carbonyl and aromatic heterocyclic groups,
a group —C(O)R₄, where R₄ is selected from —CH₂CH₃ and —C₅H₁₁,
a group —C(O)OR₅, where R₅ is a linear or branched, saturated or unsaturated C1 to C8 carbon chain,
a group —C(O)CHNH(COCH₂CH₃)R₆ where R₆ is the side chain of the amino acids selected from —CH₂—C₃N₂H₂, CH₂CH(CH₃)₂, —CH(CH₃)CH₂CH₃, —CH(CH₃)₂, —CH₂C₆H₅, —CH₂C₈NH₆, —(CH₂)₄NH₂, —CH₂C₆OH₅, —C₃H₅N,

for use as a drug for causing regression of a mammalian cancerous tumor.

The invention relates secondly to a pharmaceutical composition comprising, in a pharmaceutically acceptable vehicle, at least one compound of formula (I) for use thereof as a drug for causing regression of a mammalian cancerous tumor.

In this description, unless specified otherwise, it is understood that when a range is given, it includes the upper and lower limits of said range.

In the present text, the following terms, unless stated otherwise, are to be understood to have the following meanings:

The term “solvate” is used hereto describe a molecular complex comprising a compound of the invention and containing stoichiometric or substoichiometric amounts of one or more molecules of pharmaceutically acceptable solvent such as ethanol. The term “hydrate” is referred to when said solvent is water.

The term “allyl” refers to a functional alkene group of condensed formula H₂C═CH—CH₂—.

The term “carbonyl” refers to a double bond between a carbon atom and an oxygen atom.

The term “aromatic heterocycle” refers to monocyclic and polycyclic aromatic compounds comprising, as cyclic elements, one or more heteroatoms from O, S and/or N. Among the aromatic heterocycles, we may mention imidazole, furan, thiophene, pyrrole, purine, pyrimidine, indole and benzofuran.

The term “human” refers to a subject of either sex and at any stage of development (i.e. a neonate, infant, child, adolescent, adult).

The term “patient” refers to a hot-blooded animal, more preferably a human, waiting to receive or receiving medical care and/or which or who will be the object of a medical intervention.

“Pharmaceutically acceptable” means that the ingredients of a pharmaceutically acceptable product are compatible with one another and are harmless to the patient receiving this product.

The term “pharmaceutical vehicle” as used in this text signifies a vehicle or an inert medium used as solvent or diluent in which the pharmaceutically active agent is formulated and/or administered. Nonlimiting examples of pharmaceutical vehicles comprise creams, gels, lotions, solutions and liposomes.

The term “administration” means the delivery of the active agent or active ingredient (for example the compound of formula (I)), in a pharmaceutically acceptable composition, to the patient with a condition, a symptom and/or a disease that must be treated.

The terms “treat” and “treatment” such as used herein include to attenuate, alleviate, stop, or treat a condition, a symptom and/or a disease.

The term “prodrug” or “prodrug product” as used in the present description denotes the pharmacologically acceptable derivatives of the compounds of formula (I), which can be administered to a patient without excessive toxicity, irritation, allergic reaction, etc., which are convertible in vivo by metabolic means (for example by hydrolysis) and whose product of biotransformation in vivo generates the biologically active medicinal product. Most of the prodrugs described in the present description are characterized by increased bioavailability and are easily metabolized to compounds that are biologically active in vivo. The prodrug is administered in an inactive or much less active form than its metabolite. In the present description, the prodrugs have pharmacologic properties that are identical, similar or greater than the compound 5α-hydroxy-6β-[2-(1H-imidazol-4-yl)-ethylamino]-cholestan-3β-ol. Certain prodrugs described in the present invention, when they do not have bioavailability greater than that of the reference compound, display quicker penetration and potentially a quicker effect for use thereof for treating cancer.

The term “medicinal product” or “drug” in the present description denotes any compound or composition presented as possessing therapeutic or preventive properties with respect to human or animal diseases. By extension, a medicinal product comprises any compound or any composition usable in human beings or animals or that may be administered to them, with a view to establishing a medical diagnosis or of restoring, correcting or modifying their physiological functions by exerting a pharmacologic, immunologic, or metabolic action. The medicinal product is made up of two types of substances, an active ingredient and one or more excipients.

The term “active ingredient” denotes a compound having a pharmacologic effect and a therapeutic effect.

The term “excipient” denotes any substance other than the active ingredient in a medicinal product.

“Drug-resistant cancer” means a cancer in a patient where the proliferation of the cancer cells cannot be prevented or inhibited by means of an antineoplastic agent or a combination of antineoplastic agents usually employed for treating this cancer, at a dose acceptable for the patient. Tumors may be intrinsically resistant before chemotherapy, or resistance may be acquired during treatment of tumors initially sensitive to chemotherapy.

“Chemosensitive cancer” means a cancer in a patient that responds to the effects of an antineoplastic agent, i.e. where proliferation of the cancer cells may be prevented by means of said antineoplastic agent at a dose acceptable for the patient.

The compound of formula (I) belongs to the steroids group. The numbering of the carbon atoms of the compound of formula (I) therefore follows the nomenclature defined by IUPAC in Pure & Appl. Chem., Vol. 61, No. 10, pp. 1783-1822, 1989. The numbering of the carbon atoms of a compound belonging to the steroids group according to IUPAC is illustrated below:

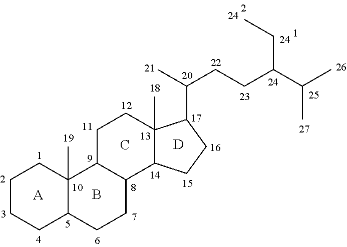

In the present description, the following abbreviations mean:
AML: acute myeloid leukemia;
Dendrogenin A: 5α-hydroxy-6β-[2-(1H-imidazol-4-yl)-ethylamino]-cholestan-3β-ol;
MCF-7: Michigan Cancer Foundation-7;
DMEM: Dulbecco's Modified Eagle Medium;
FCS: fetal calf serum;
ChEH: Cholesterol Epoxide Hydrolase;
Neuro2a: murine glioblastoma;
CTL: Control;
MTT: 3-(4,5-dimethylthiazol-2-yl)-2,5-diphenyltetrazolium bromide;
PBS: phosphate-buffered saline;
DMSO: dimethylsulfoxide;
OD: optical density or absorbance;
CT: cholestane-3β,5α,6β-triol;
OCDO: 6-oxo-cholestan-3β,5α-diol;
5,6α-EC: 5,6α-epoxycholesterol;
Tam: tamoxifen;
TLC: thin-layer chromatography;
tert-butyl: tert.-butyl or t-butyl of formula (CH₃)₃C—.

### BRIEF DESCRIPTION OF THE DRAWINGS

The invention will be better understood, and other aims, details, features and advantages thereof will become clearer from the following description of several particular embodiments of the invention, given only for purposes of illustration and nonlimiting, referring to the appended drawings.

FIG. 1 shows the pharmacokinetic profile of the compound 5α-hydroxy-6β-[2-(1H-imidazol-4-yl)-ethylamino]-cholestan-3β-yl propionate (DX107) compared to the compound Dendrogenin A (DX101).

FIG. 2 shows the pharmacokinetic profile of the compound 5α-hydroxy-6β-[2-(1H-imidazol-4-yl)ethylamino]-cholestan-3β-yl-(2-(1H-imidazol-4-yl)ethyl)carbamate (DX117) compared to the compound Dendrogenin A (DX101).

FIG. 3 shows the pharmacokinetic profile of the compound 5α-hydroxy-6β-[2-(1H-imidazol-4-yl)-ethylamino]-cholestan-3β-ethyl-carbonate (DX121) compared to the compound Dendrogenin A (DX101).

FIG. 4A shows the comparison of activity between DX107 and DX101 on the reduction of tumor growth.

FIG. 4B shows the comparison of activity between DX107 and DX101 on the animals' survival.

FIG. 5 shows the results of a study of cytotoxicity of the compound 5α-hydroxy-6β-[2-(1H-imidazol-4-yl)-ethylamino]-cholestan-3β-yl propionate on the Neuro2a cells via a trypan blue assay.

FIG. 6 shows the results of an MTT cell viability test carried out on MCF-7 mammary tumor cells in the presence of the compound 5α-hydroxy-6β-[2-(1H-imidazol-4-yl)-ethylamino]-cholestan-3β-yl propionate.

FIG. 7 shows the results for the activity of cholesterol epoxide hydrolase (ChEH) in MCF-7 cells in the presence of the compound 5α-hydroxy-6β-[2-(1H-imidazol-4-yl)-ethylamino]-cholestan-3β-yl propionate.

### DETAILED DESCRIPTION OF THE PREFERRED EMBODIMENTS

The invention relates firstly to a compound of formula (I);

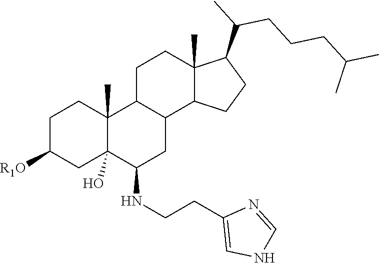

or a pharmaceutically acceptable salt of a compound of this kind, in which R₁ is selected from:
a group —C(O)NR₂R₃,
where R₂, R₃ are equivalent or different and are selected from H and a linear or branched, saturated or unsaturated C1 to C8 carbon chain optionally containing one or more substituents selected from allyl, carbonyl and aromatic heterocyclic groups,
a group —C(O)R₄, where R₄ is selected from —CH₂CH₃ and —C₅H₁₁,
a group —C(O)OR₅, where R₅ is a linear or branched, saturated or unsaturated C1 to C8 carbon chain,
a group —C(O)CHNH(COCH₂CH₃)R₆ where R₆ is the side chain of the amino acids selected from —CH₂—C₃N₂H₂, CH₂CH(CH₃)₂, —CH(CH₃)CH₂CH₃, —CH(CH₃)₂, —CH₂C₆H₅, —CH₂C₈NH₅, —(CH₂)₄NH₂, —CH₂C₆OH₅, —C₃H₅N,

for use thereof as a medicinal product.

According to one embodiment, the invention relates to a compound of formula (I);

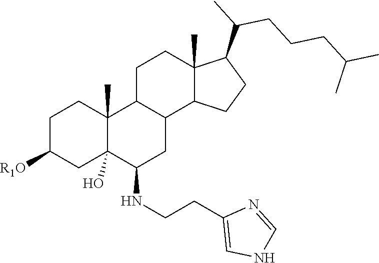

or a pharmaceutically acceptable salt of a compound of this kind, in which R₁ is selected from:
a group —C(O)NR₂R₃,
where R₂, R₃ are equivalent or different and are selected from H and a linear or branched, saturated or unsaturated C1 to C8 carbon chain optionally containing one or more substituents selected from allyl, carbonyl and aromatic heterocyclic groups,
a group —C(O)R₄, where R₄ is selected from —CH₂CH₃ and —C₅H₁₁,
a group —C(O)OR₅, where R₅ is a linear or branched, saturated or unsaturated C1 to C8 carbon chain,
a group —C(O)CHNH(COCH₂CH₃)R₆ where R₆ is the side chain of the amino acids selected from —CH₂—C₃N₂H₂, CH₂CH(CH₃)₂, —CH(CH₃)CH₂CH₃, —CH(CH₃)₂, —CH₂C₆H₅, —CH₂C₈NH₆, —(CH₂)₄NH₂, —CH₂C₆OH₅, —C₃H₅N,

for use thereof as a drug for causing regression of a mammalian cancerous tumor.

In one embodiment of the compound of formula (I), the radical R₁ is a group —C(O)R₄ (acyl group) where R₄ is the radical selected from —CH₂CH₃ and —C₅H₁₁. In this embodiment of the compound of formula (I), the radical R₄ is preferably the radical —CH₂CH₃, it is the compound 5α-hydroxy-6β-[2-(1H-imidazol-4-yl)-ethylamino]-cholestan-3β-propionate. Propionate is the same as propanoate. In another preferred embodiment of the compound of formula (I), the radical R₄ is C₅H₁₁, it is the compound 5α-hydroxy-6β-[2-(1H-imidazol-4-yl)-ethylamino]-cholestan-3β-hexanoate.

In another embodiment of the compound of formula (I), the radical R₁ is a group —C(O)OR₅ (carbonate group), where R₅ is a C1 to C8 carbon chain. In this embodiment of the compound of formula (I), the radical R₅ is preferably an ethyl or butyl carbon chain, very preferably an ethyl carbon chain.

In yet another embodiment of the compound of formula (I), the radical R₁ is a group —C(O)NR₂R₃ (carbamate group) in which R₂ and R₃ are equivalent or different and are selected from H and a linear, saturated C1 to C8 carbon chain, optionally containing an aromatic heterocyclic substituent. In a preferred embodiment of the compound of formula (I), R₂ and R₃ are selected from two ethyl radicals or the group 1-H-imidazol-4-yl. In a very preferred embodiment of the compound of formula (I), R₂ and R₃ denote an aromatic heterocyclic substituent such as the group 1-H-imidazol-4-yl.

In one embodiment of the compound of formula (I), R₁ is a group C(O)CHNH(COCH₂CH₃)R₆ where R₆ is the side chain of the amino acids selected from CH₂—C₃N₂H₂, CH₂CH(CH₃)₂, CH(CH₃)CH₂CH₃, CH(CH₃)₂, CH₂C₆H₅, CH₂C₈NH₆, (CH₂)₄NH₂, CH₂C₆OH₅, C₃H₅N.

In this embodiment of the compound of formula (I), when the radical R₁ is the group —C(O)CHNH(COCH₂CH₃)CH₂C₃N₂H, it is the compound N-propionate-L-histidine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH(CH₃)CH₂CH₃, it is the compound N-propionate-L-isoleucine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH₂CH(CH₃)₂, it is the compound N-propionate-L-leucine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)(CH₂)₄NH₂, it is the compound N-propionate-L-lysine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH₂C₆H₅, it is the compound N-propionate-L-phenylalanine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH₂C₈NH₆, it is the compound N-propionate-L-tryptophan 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH₂C₆H₄OH, it is the compound N-propionate-L-tyrosine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)CH(CH₃)₂, it is the compound N-propionate-L-valine 5α-hydroxy-6β-[2-(1H-imidazol-4-yl)-ethylamino]-cholestan-3β-yl ester.

In this embodiment of the compound of formula (I), when the radical R₁ is the group —C(O)CHNH(COCH₂CH₃)C₃H₅N, it is the compound N-propionate-L-proline α-hydroxy-6β-[2-(1H-imidazol-4-yl)-ethylamino]-cholestan-3β-yl ester.

The preferred compounds according to the invention are selected from:
5α-hydroxy-6β-[2-(1H-imidazol-4-yl)-ethylamino]-cholestan-3β-propionate;
5α-hydroxy-6β-[2-(1H-imidazol-4-yl)-ethylamino]-cholestan-3β-hexanoate;
5α-hydroxy-6β-[2-(1H-imidazol-4-yl)-ethylamino]-cholestan-3β-yl-ethyl carbonate;
5α-hydroxy-6β-[2-(1H-imidazol-4-yl)-ethylamino]-cholestan-3β-yl butyl carbonate;
5α-hydroxy-6β-[2-(1H-imidazol-4-yl)-ethylamino]-cholestan-3β-yl 1-H-imidazol-4-yl ethyl carbamate;
5α-hydroxy-6β-[2-(1H-imidazol-4-yl)-ethylamino]-cholestan-3β-yl diethyl carbamate;
N-propionate-L-histidine 5α-hydroxy-6β-[2-(1H-imidazol-4-yl)-ethylamino]-cholestan-3β-yl ester;
N-propionate-L-leucine 5α-hydroxy-6β-[2-(1H-imidazol-4-yl)-ethylamino]-cholestan-3β-yl ester;
N-propionate-L-isoleucine 5α-hydroxy-6β-[2-(1H-imidazol-4-yl)-ethylamino]-cholestan-3β-yl ester;
N-propionate-L-valine 5α-hydroxy-6β-[2-(1H-imidazol-4-yl)-ethylamino]-cholestan-3β-yl ester;
N-propionate-L-phenylalanine 5α-hydroxy-6β-[2-(1H-imidazol-4-yl)-ethylamino]-cholestan-3β-yl ester;
N-propionate-L-tryptophan 5α-hydroxy-6β-[2-(1H-imidazol-4-yl)-ethylamino]-cholestan-3β-yl ester;
N-propionate-L-lysine 5α-hydroxy-6β-[2-(1H-imidazol-4-yl)-ethylamino]-cholestan-3β-yl ester;
N-propionate-L-tyrosine 5α-hydroxy-6β-[2-(1H-imidazol-4-yl)-ethylamino]-cholestan-3β-yl ester. imidazol-4-yl)-ethylamino]-cholestan-3β-yl ester,
N-propionate-L-proline α-hydroxy-6β-[2-(1H-imidazol-4-yl)-ethylamino]-cholestan-3β-yl ester. imidazol-4-yl)-ethylamino]-cholestan-3β-yl ester.

The very preferred compounds according to the invention are selected from:
5α-hydroxy-6β-[2-(1H-imidazol-4-yl)-ethylamino]-cholestan-3β-propionate;
5α-hydroxy-6β-[2-(1H-imidazol-4-yl)-ethylamino]-cholestan-3β-hexanoate;
5α-hydroxy-6β-[2-(1H-imidazol-4-yl)-ethylamino]-cholestan-3β-yl-ethyl carbonate;
5α-hydroxy-6β-[2-(1H-imidazol-4-yl)-ethylamino]-cholestan-3β-yl butyl carbonate;
5α-hydroxy-6β-[2-(1H-imidazol-4-yl)-ethylamino]-cholestan-3β-yl 1-H-imidazol-4-yl ethyl carbamate;
5α-hydroxy-6β-[2-(1H-imidazol-4-yl)-ethylamino]-cholestan-3β-yl diethyl carbamate;
N-propionate-L-tyrosine 5α-hydroxy-6β-[2-(1H-imidazol-4-yl)-ethylamino]-cholestan-3β-yl ester.

According to one embodiment, the compound of formula (I) is intended to be used as a drug in the treatment of breast cancer, prostate cancer, colorectal cancer, lung cancer, bladder cancer, skin cancer, cancer of the uterus, cervical cancer, mouth cancer, brain cancer, stomach cancer, liver cancer, throat cancer, cancer of the larynx, cancer of the esophagus, bone cancer, ovarian cancer, cancer of the pancreas, cancer of the kidney, cancer of the retina, cancer of the sinus, cancer of the nasal cavities, testicular cancer, cancer of the thyroid, cancer of the vulva, in the treatment of lymphoma, non-Hodgkin lymphoma, Hodgkin lymphoma, leukemia, acute myeloid leukemia or acute lymphocytic leukemia, multiple myeloma, Merkel cell carcinoma or mesothelioma.

According to one embodiment, the cancer is an acinar cell adenocarcinoma, an acinar cell carcinoma, an acro-lentiginous melanoma, an actinic keratosis, an adenocarcinoma, a cystic adenoid carcinoma, an adenosquamous carcinoma, an adnexal carcinoma, an adrenal rest tumor, an adrenocortical carcinoma, an aldosterone secreting carcinoma, an alveolar sarcoma of the soft part, an ameloblastic carcinoma of the thyroid, an angiosarcoma, an apocrine carcinoma, an Askin tumor, astrocytoma, a basal cell carcinoma, a basaloid carcinoma, a basosquamous carcinoma, a bile duct cancer, a bone marrow cancer, a botryoid sarcoma, a bronchioalveolar carcinoma, a bronchogenic adenocarcinoma, a bronchogenic carcinoma, an ex adenoma pleomorphic carcinoma, a chloroma, a cholangiocellular carcinoma, a chondrosarcoma, a choriocarcinoma, a carcinoma of the choroid plexus, a clear cell adenocarcinoma, a colon cancer, a comedocarcinoma, a cortisol producing carcinoma, a cylindrical cell carcinoma, a differentiated liposarcoma, a ductal adenocarcinoma of the prostate, a ductal carcinoma, a ductal carcinoma in situ, a duodenal cancer, an eccrine carcinoma, an embryonic carcinoma, a carcinoma of the endometrium, a stromal carcinoma of the endometrium, an epithelioid sarcoma, a Ewing sarcoma, an exophytic carcinoma, fibroblastic sarcoma, a fibrocarcinoma, a fibrolamellar carcinoma, a fibrosarcoma, follicular carcinoma of the thyroid, a cancer of the gallbladder, a gastric adenocarcinoma, a giant cell carcinoma, a giant cell sarcoma, a giant cell osseous tumor, a glioma, a glioblastoma multiforme, a granulocytic carcinoma, a head and neck cancer, a hemangioma, a hemangiosarcoma, a hepatoblastoma, a hepatocellular carcinoma, a Hurthle cell carcinoma, an ileal cancer, an infiltrating lobular carcinoma, an inflammatory breast carcinoma, an intraductal carcinoma, an intraepidermal carcinoma, a cancer of the jejunum, a Kaposi sarcoma, a Krukenberg tumor, a Kulchitsky cell carcinoma, a Kupffer cell sarcoma, a large-cell carcinoma, a cancer of the larynx, a lentigo maligna melanoma, a liposarcoma, a lobular carcinoma, a lobular carcinoma in situ, a lymphoepithelioma, a lymphosarcoma, a malignant melanoma, a medullar carcinoma, a medullar carcinoma of the thyroid, a medulloblastoma, a meningeal carcinoma, a micropapillar carcinoma, a mixed cell sarcoma, a mucinous carcinoma, a muco-epidermoid carcinoma, a mucosal melanoma, a myxoid liposarcoma, a myxosarcoma, a nasopharyngeal carcinoma, nephroblastoma, a neuroblastoma, a nodular melanoma, a non-clear cell kidney cancer, a nonsmall cell lung cancer, an oat cell carcinoma, an ocular melanoma, a mouth cancer, an osteoid carcinoma, an osteosarcoma, an ovarian cancer, a Paget carcinoma, a pancreatoblastoma, a papillar adenocarcinoma, a papillar carcinoma, a papillar carcinoma of the thyroid, a pelvic cancer, a periampullary carcinoma, a phyllodes tumor, a cancer of the pituitary gland, pleomorphic liposarcoma, a pleuropulmonary blastoma, a primary intraosseous carcinoma, a cancer of the rectum, a renal cell carcinoma, a retinoblastoma, a rhabdomyosarcoma, a round cell liposarcoma, a cicatricial cancer, a schistosomal bladder cancer, a schneiderian carcinoma, a sebaceous carcinoma, a ring cell carcinoma, a cancer of the skin, a small cell lung cancer, a small cell osteosarcoma, a sarcoma of the soft tissues, a fusiform cell sarcoma, an epidermoid carcinoma, a stomach cancer, a superficial spreading melanoma, a synovial sarcoma, a telangiectatic sarcoma, a carcinoma of the terminal duct, a testicular cancer, a thyroid cancer, a transitional cell carcinoma, a tubular carcinoma, a tumorigenic melanoma, an undifferentiated carcinoma, an adenocarcinoma of the urethra, a bladder cancer, a cancer of the uterus, a uterine body carcinoma, a melanoma of the uterus, a cancer of the vagina, a verrucous carcinoma, a villous carcinoma, a well-differentiated liposarcoma, a Wilms tumor or germ cell tumors.

In a preferred embodiment, the compound of formula (I) is intended to be used as a drug in the treatment of breast cancer, myeloid leukemia and melanoma in mammals.

According to one embodiment, the compound is intended to be used as a drug in the treatment of a chemosensitive cancer.

According to a particularly preferred embodiment, the compound of formula (I) is intended to be used as a drug in the treatment of a drug-resistant cancer.

According to one embodiment, the drug-resistant cancer is a hematologic or blood cancer, such as leukemia, in particular acute myeloid leukemia or acute lymphocytic leukemia, lymphoma, in particular non-Hodgkin lymphoma and multiple myeloma.

According to one embodiment, the cancer is drug-resistant to daunorubicin, cytarabine, fluorouracil, cisplatin, all-trans retinoic acid, arsenic trioxide, bortezomib or a combination thereof.

The specific C3 prodrugs of the compound Dendrogenin A described in the present description display pharmacologic activity comparable to or greater than Dendrogenin A. Dendrogenin A is eliminated quickly in vivo by the body. The specific C3 prodrugs according to the invention have bioavailability greater than Dendrogenin A and are easily metabolized to biologically active compounds in vivo. Consequently, the therapeutic effect of Dendrogenin A is prolonged in the patient's body when a specific C3 prodrug described in the present description is used in vivo.

All references to the compounds of formula (I) comprise the references to the salts, to multi-component complexes and liquid crystals thereof. All references to the compounds of formula (I) also comprise the references to the polymorphs and the usual crystals thereof.

The compound according to the invention may be in the form of pharmaceutically acceptable salts. A pharmaceutically acceptable salt of the compound of formula (I) comprises the acid addition thereof.

Suitable acid salts are formed from acids that form nontoxic salts. For example, the salts are selected from: acetate, adipate, benzoate, bicarbonate, carbonate, bisulfate, sulfate, camphosulfonate, borate, camsylate, citrate, cyclamate, edisylate, esylate, formate, fumarate, gluceptate, gluconate, glucuronate, hexafluorophosphate, hibenzate, chloride hydrochloride, hydrobromide, bromide, hydriodide, iodide, isethionate, lactate, malate, maleate, malonate mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate salts, tosylate, trifluoroacetate and xinafoate. Preferably, the pharmaceutically acceptable salt of the compound of formula (I) is formed from lactate.

The pharmaceutically acceptable salts of the compounds of formula (I) may be prepared by one or more of the following three methods:
(i) by reacting the compound of formula (I) with the desired acid;
(ii) by eliminating a labile protective group in an acid or basic medium of a suitable precursor of the compound of formula (I) or by ring opening of a suitable cyclic precursor, for example a lactone or a lactam, using the desired acid; or
iii) by converting a salt of the compound of formula (I) into another by reaction with an acid or by means of a suitable ion exchange column.

These three reactions are generally carried out in solution. The salt obtained may precipitate and may be collected by filtration or may be recovered by solvent evaporation. The degree of ionization of the salt obtained may vary from completely ionized to almost unionized.

The invention relates secondly to a pharmaceutical composition comprising, in a pharmaceutically acceptable vehicle, at least one compound according to the invention as described above for use thereof as a drug for causing regression of a mammalian cancerous tumor.

According to one embodiment, the pharmaceutical composition further comprises at least one other therapeutic agent.

According to a preferred embodiment, this other therapeutic agent is an antineoplastic agent.

According to one embodiment, the antineoplastic agent is an agent that damages DNA such as camptothecin, irinotecan, topotecan, amsacrine, etoposide, phosphate etoposide, teniposide, cisplatin, carboplatin, oxaliplatin, cyclophosphamide, chlorambucil, chlormethine, busulfan, treosulfan or thiotepa, an antitumor antibiotic such as daunorubicin, doxorubicin, epirubicin, idarubicin, mitoxantrone, valrubicin, actinomycin D, mitomycin, bleomycin or plicamycin, an antimetabolite such as 5-fluorouracil, cytarabine, fludarabine or methotrexate, an antimitotic such as paclitaxel, docetaxel, vinblastine, vincristine, vindesine or vinorelbine, or various antineoplastic agents such as bortezomib, all-trans retinoic acid, arsenic trioxide, or a combined product thereof.

According to one embodiment, the pharmaceutical composition is used in cancer treatment in a patient with a tumor that is drug-resistant to said antineoplastic agent when it is not administered in combination with a compound according to the invention.

According to one embodiment, the pharmaceutical composition is used in cancer treatment in a patient with a tumor that is chemosensitive to said antineoplastic agent, and the dose of the antineoplastic agent administered to said patient in combination with a compound according to the invention or a pharmaceutically acceptable salt thereof is lower than the dose of the antineoplastic agent when it is not administered in combination with a compound according to the invention. In particular, the dose of the antineoplastic agent administered to said patient in combination with a compound according to the invention or a pharmaceutically acceptable salt thereof is lower than the dose of the antineoplastic agent administered alone, without another active ingredient.

The pharmaceutical composition according to the invention may also further comprise other active therapeutic compounds commonly used in the treatment of the aforementioned pathology.

According to one embodiment, the pharmaceutical composition comprises the compound according to the invention as the only therapeutic agent.

According to one embodiment, the pharmaceutical composition comprises the compound of formula (I) administered to the patient as an active therapeutic agent.

According to one embodiment, the pharmaceutical composition comprises the compound of formula (I) administered to the patient in combination with at least one other active therapeutic agent.

According to one embodiment, the pharmaceutical composition of the invention may be administered by all routes, in particular by the routes: intradermal, intramuscular, intraperitoneal, intravenous, or subcutaneous, pulmonary, transmucosal (oral, intranasal, intravaginal, rectal), inhalation by nasal spray, using a formulation as tablet, capsule, solution, powder, gel, particle; and contained in a syringe, an implanted device, an osmotic pump, a cartridge, a micropump; or any other means assessed by a qualified professional, well known in the art. Specific administration at a site may be carried out, for example by the following routes: intratumoral, intraarticular, intrabronchial, intra-abdominal, intracapsular, intracartilaginous, intracavitary, intracerebellar, intracerebroventricular, intracolic, intracervical, intragastric, intrahepatic, intracardiac, intraosteal, intrapelvic, intrapericardial, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal or transdermal in a suitable dosage comprising the usual nontoxic and pharmaceutically acceptable vehicles. Preferably, the pharmaceutical composition is in a suitable form to be administered intravenously, subcutaneously, intraperitoneally, or orally. The oral route is particularly preferred.

Besides hot-blooded animals such as mice, rats, dogs, cats, sheep, horses, cows and monkeys, the compound of the invention is also effective in humans.

According to one embodiment, the pharmaceutical compositions for administration of the compounds of this invention may be presented in the form of unit doses and may be prepared by one of the methods that are well known in the prior art. All the methods comprise the step consisting of combining the active ingredient with the vehicle, which constitutes one or more accessory ingredients. In general, the pharmaceutical compositions are prepared by combining the active ingredient with a liquid vehicle or a finely divided solid vehicle, or both, and then, if necessary, forming the product in the desired formulation. In the pharmaceutical composition, the compound of the active ingredient is included in a sufficient amount to produce the desired effect on the process or the state of the diseases. The pharmaceutical compositions containing the active ingredient may be in a form suitable for oral usage, for example in the form of tablets, pastilles, aqueous or oily suspensions, dispersible powders or granules, emulsions, capsules, syrups, elixirs, solutions, buccal patches, oral gel, chewing gum, chewable tablets, effervescent powder and effervescent tablets. The pharmaceutical compositions containing the active ingredient may be in the form of an aqueous or oily suspension.

According to one embodiment, the aqueous suspensions contain the active substances mixed with suitable excipients for making aqueous suspensions. These excipients are suspending agents, for example carboxymethylcellulose sodium, methylcellulose, hydroxy-propylmethylcellulose, sodium alginate, polyvinyl-pyrrolidone, tragacanth and acacia; the dispersants or wetting agents may be a natural phosphatide, for example lecithin, or condensation products of an alkylene oxide with fatty acids, for example polyoxyethylene stearate, or condensation products of ethylene oxide with long-chain aliphatic alcohols, for example heptadecaethylene-oxyketanol, or condensation products of ethylene oxide with partial esters derived from fatty acids and a hexitol, such as polyoxyethylene sorbitol monooleate, or condensation products of ethylene oxide with partial esters derived from fatty acids and hexitol anhydrides, for example polyethylene sorbitol monooleate. The aqueous suspensions may also contain one or more preservatives, for example ethyl, or n-propyl, p-hydroxybenzoate, one or more colorants, one or more flavorings, and one or more sweeteners, such as sucrose or saccharin.

According to one embodiment, the oily suspensions may be formulated by suspending the active ingredient in a vegetable oil, for example peanut oil, olive oil, sesame oil or coconut oil, or in a mineral oil such as liquid paraffin. The oily suspensions may contain a thickener, for example beeswax, hard paraffin or cetyl alcohol. Sweeteners such as those mentioned above and flavorings may be added to obtain an oral preparation with a pleasant taste. These compositions may be preserved by adding an antioxidant such as ascorbic acid. The powders and dispersible granules that are suitable for preparing an aqueous suspension by adding water deliver the active ingredient mixed with a dispersant or wetting agent, a suspending agent and one or more preservatives.

The syrups and elixirs may be formulated with sweeteners, for example glycerol, propylene glycol, sorbitol or sucrose. These formulations may also contain an emollient, a preservative, flavorings and colorants.

The pharmaceutical compositions according to the invention may be in the form of an aqueous or oily suspension for sterile injection. This suspension may be formulated according to the known art using the suitable dispersants or wetting agents and the suspending agents that are mentioned above. The injectable sterile preparation may also be a sterile solution or a suspension injectable in a diluent or an acceptable nontoxic solvent by the parenteral route, for example a solution in 1,3-butanediol. The acceptable vehicles and solvents that may be used comprise; water, Ringer fluid and isotonic solution of sodium chloride. Furthermore, sterile fixed oils are used conventionally as solvent or suspension medium. For this purpose, any fixed oil may be used, including the synthetic mono- or diglycerides. Furthermore, the fatty acids such as oleic acid are used in the preparation of the injectable products.

The pharmaceutical compositions according to the invention may also be administered in the form of suppositories for rectal administration of the medicinal product. These compositions may be prepared by mixing the medicinal product with a suitable nonirritant excipient that is solid at ordinary temperature but liquid at the rectal temperature and that will therefore melt in the rectum to release the medicinal product. These materials comprise cocoa butter and polyethylene glycols.

Furthermore, the pharmaceutical compositions according to the invention may be administered by the ocular route by means of solutions or ointments. Moreover, the transdermal administration of the compounds in question may be carried out by means of iontophoretic patches and others. Creams, ointments, gels, solutions or suspensions are used for topical application.

In the treatment of a mammal with or at risk of developing a cancer, a suitable dosage of the pharmaceutical composition according to the invention may generally be of about 0.1 to 50 000 micrograms (μg) per kg of the patient's body weight per day, which may be administered in single or multiple doses. The dosage level will preferably be from about 1000 to about 40 000 μg/kg per day, depending on many factors such as the severity of the cancer to be treated, the subject's age and relative state of health, and the route and form of administration. For oral administration, this composition may be supplied in the form of tablets containing 1000 to 100000 micrograms of each of the active ingredients, in particular 1000, 5000, 10000, 15000, 20000, 25000, 50000, 75000, 100000 micrograms of each active ingredient. This composition may be administered according to a scheme of 1 to 4 times per day, for example once or twice daily. The posologic regime may be adjusted to provide an optimal therapeutic response.

The invention also discloses hereunder methods of manufacture of the compounds of formula (I).

### EXAMPLES

Various experiments were undertaken to evaluate the characteristics of the compounds of formula (I).

The term “ambient temperature” used in the following examples is to be interpreted as being a temperature between 10 and 40 degrees Celsius (° C.), for example between 15° C. and 30° C. and preferably about 20° C.

The preferred compounds according to the invention corresponding to general formula I, whose synthesis and activity are described hereunder, are as follows:

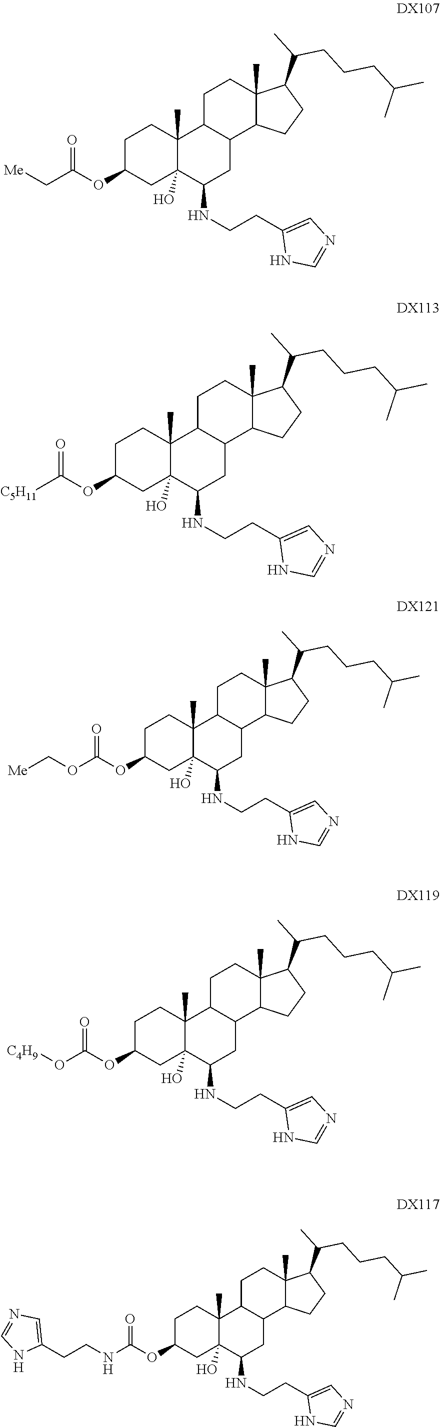

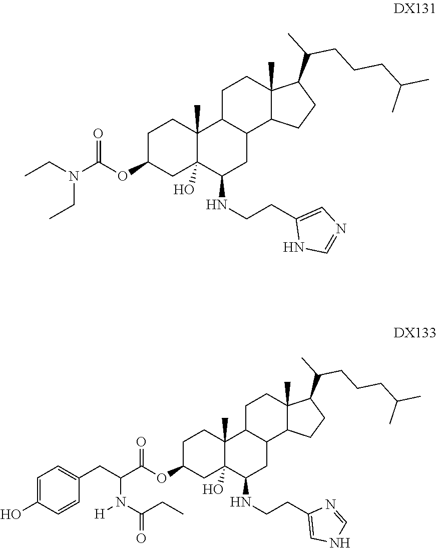

The other compounds within the scope of the general formula, not exemplified, form an integral part of the compounds according to the invention.

### Example 1: Synthesis of the Compound of Formula (I) 5α-hydroxy-6β-[2-(1H-imidazol-4-yl)ethylamino]cholestan-3β-yl propionate (Called DX107, in the Basic Form)

The first step is synthesis of the compound cholestan-3β-propionate comprising the following steps:

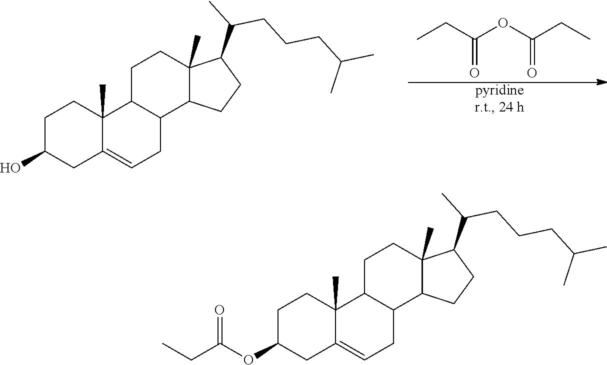

In a 100 mL flask with ground neck, 10.0 mL of anhydrous pyridine (123.6 mmol) is added to 4.00 g of cholesterol (10.3 mmol). 7.07 g of propionic anhydride (54.3 mmol) is added and the whole is stirred at room temperature for 24 hours. At the end of 24 hours, a white precipitate appears in the mixture. The reaction is stopped by adding 50 mL of methanol (MeOH) and a large amount of white precipitate is obtained. The solution is filtered and the precipitate is washed with MeOH. The procedure gives 4.50 g of a white powder corresponding to cholestan-3β-propionate (yield 89%). The 3β-propionate-cholestane is used as it is without additional purification.

¹H-NMR (500 MHz, CDCl₃): δ (ppm) 5.37-5.36 (d, 1H), 4.64-4.58 (m, 1H), 2.32-2.27 (m, 4H), 2.02-1.95 (t, 2H), 1.86-1.79 (m, 3H), 1.61-1.81 (m, 27H), 0.92-0.90 (d, 3H), 0.87-0.85 (d, 6H), 0.67 (s, 3H).

The second step consists of the synthesis, starting from cholestan-3β-propionate, of the compound 5,6α-epoxycholestan-3β-propionate as follows:

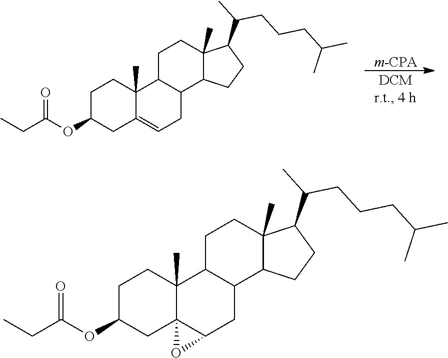

Meta-chloroperoxybenzoic acid (m-CPBA) (at 77%, 2.67 g, 11.9 mmol) is dissolved in dichloromethane (60 mL) and added dropwise in the space of 1 h to a mixture of cholestan-3β-propionate (4.00 g, 9.03 mmol) dissolved in dichloromethane (20 mL). Stirring is maintained at room temperature for three hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 4.08 g of a white powder corresponding to the mixture of the two isomers: 5,6α-epoxycholestan-3β-propionate (73%) and 5,6β-epoxycholestan-3β-propionate (27%). The white powder is used as it is without additional purification.

¹H-NMR (500 MHz, CDCl₃): δ (ppm) 4.99-4.93 (q, 1H), 2.89-2.88 (d, 1H), 2.31-2.25 (m, 2H), 2.18-2.13 (t, 1H) 2.00-1.77 (m, 4H), 1.70-0.94 (m, 29H), 0.89-0.88 (d, 3H), 0.86-0.85 (d, 6H), 0.60 (s, 3H).

The third step consists of synthesizing 5α-hydroxy-6β-[2-(1H-imidazol-4-yl)ethylamino]cholestane-3β-propionate (DX107 in basic form) as follows:

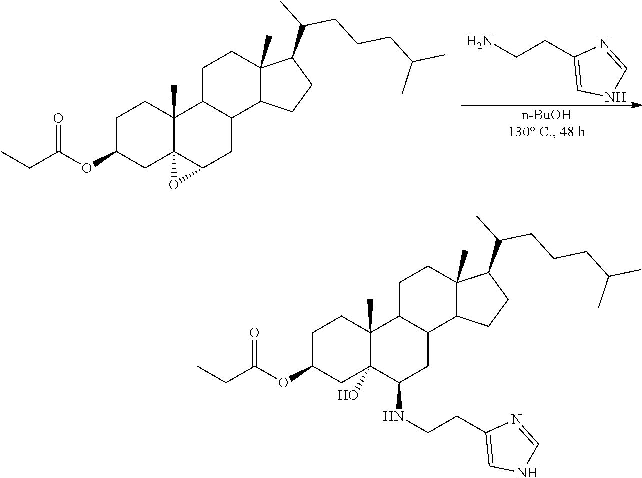

Histamine in its basic form (1.44 g, 13.0 mmol) is added after complete dissolution to 3.00 g of 5,6α-epoxycholestan-3β-propionate (at 73%, 4.6 mmol) under 30 ml of butanol. The reaction mixture is stirred under reflux for 48 hours. The progress of the reaction is monitored by thin-layer chromatography (TLC) to monitor the conversion of the 5,6α-epoxycholestan-3β-propionate. After cooling, the reaction mixture is diluted in 24 mL of methyl tert-butyl ether, the organic phase is washed twice with 24 mL of water and then twice with saturated NaCl solution. The organic phase is dried over anhydrous MgSO₄. The crude reaction product is purified in a silica gel chromatographic column on an automatic purifier. The eluent used is a dichloromethane/ethyl acetate mixture from 100-0% to 0-100%. A white powder of 1.20 g of 5α-hydroxy-6β-[2-(1H-imidazol-4-yl)ethylamino]cholestan-3β-yl propionate is obtained, corresponding to 46% yield.

¹H-NMR (500 MHz, MeOD-4d): δ (ppm) 7.60 (s, 1H), 6.85 (s, 1H), 5.21-5.15 (q, 1H), 2.94-2.89 (m, 1H), 2.77-2.69 (m, 3H), 2.39 (s, 1H), 2.34-2.29 (m, 2H), 2.12-2.07 (t, 1H), 2.01-1.99 (bd, 2H), 1.89-1.79 (m, 2H), 1.70-1.01 (m, 29H), 0.94-0.93 (d, 3H), 0.9-0.89 (dd, 6H), 0.69 (s, 3H).

### Example 2: Preparation of a Dilactate Salt of 5α-hydroxy-6β-[2-(1H-imidazol-4-yl)ethylamino]cholestan-3β-yl propionate (DX107 in the Dilactate Form)

A dilactate salt of the compound 5α-hydroxy-6β-[2-(1H-imidazol-4-yl)ethylamino]cholestan-3β-yl propionate was prepared as follows:

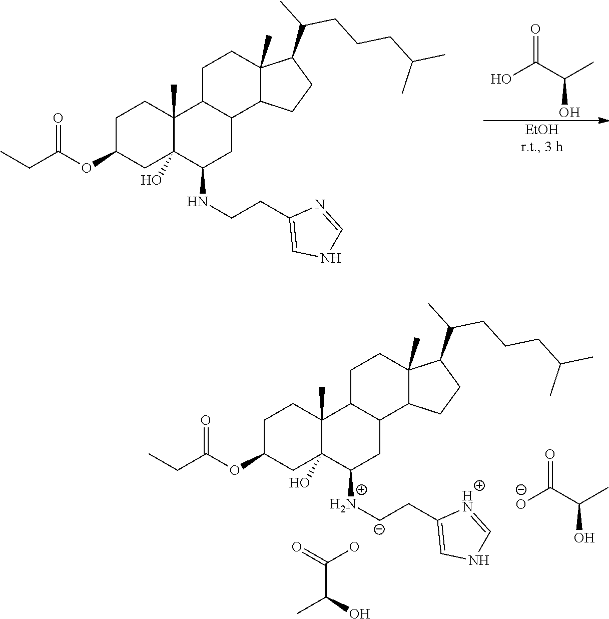

384 mg of lactic acid (4.3 mmol) was added to a solution of 1.20 g of 5α-hydroxy-6β-[2-(1H-imidazol-4-yl)ethylamino]cholestan-3β-yl propionate (2.1 mmol) in 20 mL of anhydrous ethanol with stirring. Stirring was maintained at room temperature for three hours. Vacuum evaporation of the organic solvent gives a white powder of 1.58 g of 5α-hydroxy-6β-[2-(1H-imidazol-4-yl)ethylamino]cholestan-3β-yl propionate dilactate.

¹H-NMR (500 MHz, MeOD-4d): δ (ppm) 7.61 (s, 1H), 6.85 (s, 1H), 5.04-4.98 (q, 1H), 3.93-3.89 (q, 2H), 3.43-3.42 (q, 1H), 3.25-3.20 (m, 1H), 3.12-3.06 (q, 1H), 2.15-2.09 (m, 3H), 1.87-1.85 (d, 1H), 1.69-1.67 (m, 3H), 1.61-1.05 (m, 4H), 1.41-0.82 (m, 33H), 0.76-0.75 (d, 3H), 0.69-0.68 (d, 6H), 0.57 (s, 3H).

### Example 3: Synthesis of the Compound of Formula (I) 5α-hydroxy-6β-[2-(1H-imidazol-4-yl)-ethylamino]-cholestan-3β-yl hexanoate (Called DX113, in the Basic Form)

The first step is synthesis of the compound cholestan-3β-hexanoate comprising the following steps:

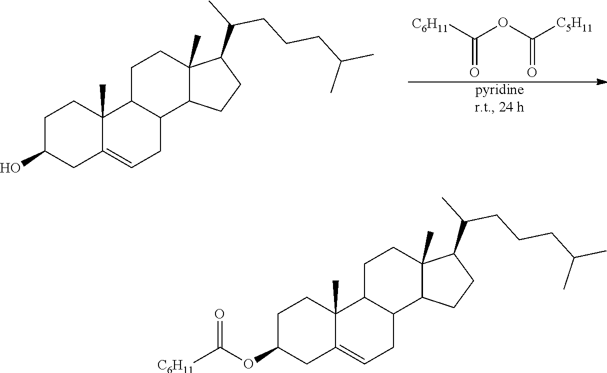

In a 100 mL flask with ground neck, 4.00 g (10.3 mmol) of cholesterol is dissolved in 10 mL of anhydrous pyridine (123.6 mg) and 11.64 g of hexanoic anhydride (54.3 mmol) is added. The whole is stirred at room temperature for 24 hours. At the end of 24 hours, a white precipitate appears in the mixture. The reaction is stopped by adding 50 mL of methanol (MeOH) and a large amount of white precipitate is obtained. The solution is filtered and the precipitate is washed with MeOH. The procedure gives 4.95 g of a white powder corresponding to cholestan-3β-hexanoate (yield 99%). The cholestan-3β-hexanoate is used as it is without additional purification.

¹H-NMR (500 MHz, CDCl₃): δ (ppm) 5.38 (s, 1H), 4.64-4.58 (m, 1H), 2.32-2.25 (m, 4H), 2.02-1.95 (m, 2H), 1.86-1.84 (m, 3H), 1.63-0.86 (m, 42H), 0.67 (s, 3H).

The second step consists of synthesizing, starting from cholestan-3β-hexanoate, the compound 5,6α-epoxycholestan-3β-hexanoate as follows:

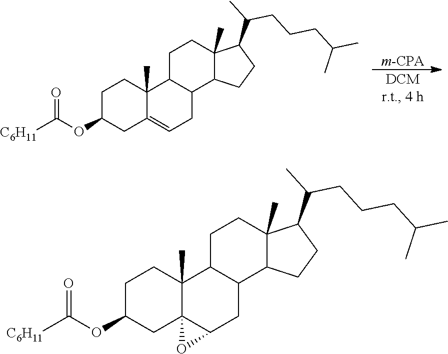

Meta-chloroperoxybenzoic acid (m-CPBA) (at 77%, 2.95 g, 17.1 mmol) is dissolved in dichloromethane (60 mL) and added dropwise in the space of 1 hour to a solution of cholestan-3β-hexanoate (4.90 g, 10.1 mmol) in dichloromethane (20 mL). Stirring is maintained at room temperature for 3 hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 5.06 g of a white powder corresponding to 5,6α-epoxycholestan-3β-hexanoate (70%) and 5,6β-epoxycholestan-3β-hexanoate (30%). The 5,6α-epoxycholestan-3β-hexanoate is used as it is without additional purification.

¹H-NMR (500 MHz, CDCl₃): δ (ppm) 4.99-4.93 (q, 1H), 2.89-2.88 (d, 1H), 2.26-2.23 (m, 2H), 2.18-2.13 (t, 1H), 2.00-0.85 (m, 48H), 0.60 (s, 3H).

The third step consists of synthesizing 5α-hydroxy-6β-[2-(1H-imidazol-4-yl)ethylamino]-cholestane-3β-hexanoate as follows (DX113 in the basic form):

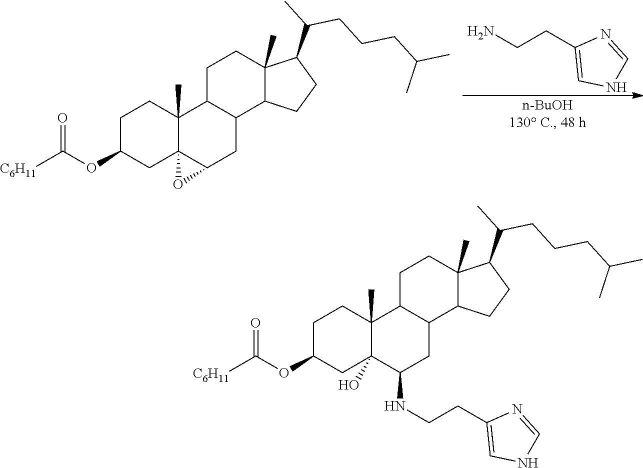

Histamine in its basic form (1.25 g, 11.2 mmol) is added to a butanolic solution (40 mL) of 5,6α-epoxycholestan-3β-hexanoate (at 70% purity, 4.0 g, 5.6 mmol). The reaction mixture is stirred under reflux for 48 hours. The progress of the reaction is monitored by thin-layer chromatography (TLC) to monitor the conversion of the 5,6α-epoxycholestan-3β-hexanoate. After cooling, the reaction mixture is diluted in 40 mL of methyl tert-butyl ether, the organic phase is washed three times with 40 mL of water and then once with 40 ml of saturated NaCl solution. The organic phase is dried over anhydrous MgSO₄. The crude reaction product is purified in a silica gel chromatographic column on an automatic purifier. The eluent used is a dichloromethane/ethyl acetate mixture from 100-0% to 0-100%. A white powder of 1.71 g of 5α-hydroxy-6β-[2-(1H-imidazol-4-yl)ethylamino]cholestane-3β-hexanoate (50% yield) is obtained.

¹H-NMR (500 MHz, MeOD-4d): δ (ppm) 7.59 (s, 1H), 6.85 (s, 1H), 5.22-5.16 (q, 1H), 2.94-2.89 (m, 1H), 2.77-2.67 (m, 3H), 2.39 (s, 1H), 2.30-2.28 (t, 2H), 2.14-2.10 (t, 1H), 2.01-1.99 (bd, 1H), 1.88-0.89 (m, 47H), 0.69 (s, 3H).

### Example 4: Preparation of a Dilactate Salt of the Compound 5α-hydroxy-6β-[2-(1H-imidazol-4-yl)ethylamino]cholestan-3β-yl hexanoate (DX113 in the Dilactate Form)

A dilactate salt of the compound 5α-hydroxy-6β-[2-(1H-imidazol-4-yl)-ethylamino]-cholestan-3β-yl hexanoate was prepared as follows:

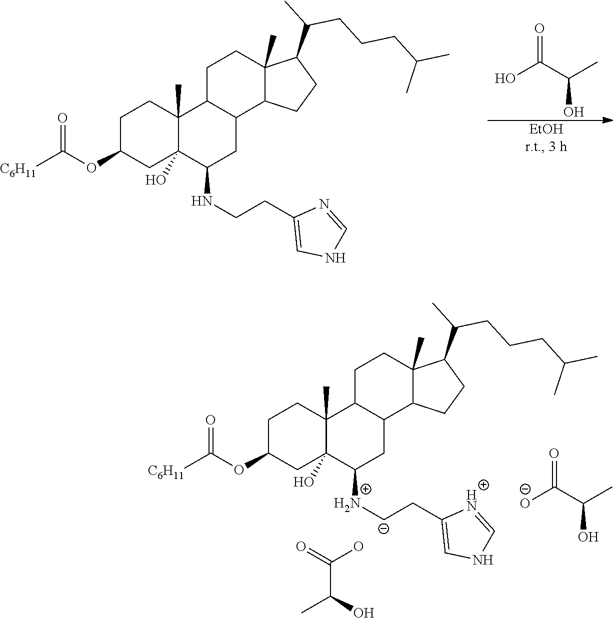

552 mg of lactic acid (6.15 mmol) was added to a solution of 1.87 g of 5α-hydroxy-6β-[2-(1H-imidazol-4-yl)-ethylamino]-cholestan-3β-yl hexanoate (3.06 mmol) in 5 mL of anhydrous ethanol with stirring. Stirring was maintained at room temperature for 3 hours. Vacuum evaporation of the organic solvent gives a white powder of 2.42 g of 5α-hydroxy-6β-[2-(1H-imidazol-4-yl)-ethylamino]-cholestan-3β-yl hexanoate dilactate.

¹H-NMR (500 MHz, MeOD-4d): δ (ppm) 7.56 (s, 1H), 6.78 (s, 1H), 4.93-4.87 (q, 1H), 3.84-3.82 (d, 2H), 3.33-3.32 (m, 1H), 3.13 (bs, 1H), 3.02 (bs, 1H), 2.75-2.69 (m, 3H), 2.08-2.05 (m, 3H), 1.78-1.76 (d, 1H), 1.61-0.59 (m, 50H), 0.48 (s, 3H).

### Example 5: Synthesis of the Compound of Formula (I) 5α-hydroxy-6β-[2-(1H-imidazol-4-yl)ethylamino]cholestan-3β-yl ethyl carbonate (Called DX121, in the Basic Form)

The first step consists of synthesizing, starting from the commercial product cholestan-3β-yl ethyl carbonate, the compound 5,6α-epoxycholestan-3β-yl ethyl carbonate as follows:

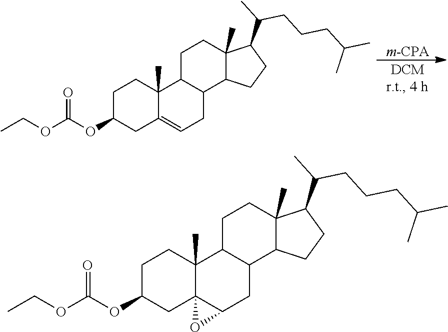

Meta-chloroperoxybenzoic acid (m-CPBA) (at 77%, 1.31 g, 5.8 mmol) is dissolved in dichloromethane (30 mL) and added dropwise in the space of 30 minutes to a mixture of cholestan-3β-yl ethyl carbonate (2.02 g, 4.4 mmol) dissolved in dichloromethane (15 mL). Stirring is maintained at room temperature for three hours. The reaction mixture is washed twice with an aqueous solution of sodium sulfite (10 wt %), twice with a saturated solution of NaHCO₃ and once with saturated NaCl solution. The organic phase is dried over anhydrous MgSO₄. Vacuum evaporation of the organic solvent gives 2.09 g of a white powder corresponding to the mixture of the two isomers: 5,6α-epoxycholestan-3β-yl ethyl carbonate (76%) and 5,6β-epoxycholestan-3β-yl ethyl carbonate (24%). The white powder is used as it is without additional purification.

¹H-NMR (500 MHz, CDCl): δ (ppm) 4.85-4.78 (q, 1H), 2.90-2.89 (d, 1H), 2.23-2.19 (t, 1H), 2.09-2.04 (m, 1H), 1.98-1.87 (m, 2H), 1.84-0.93 (m, 32H), 0.89-0.87 (d, 3H), 0.86-0.84 (d, 6H), 0.60 (s, 3H).

The second step consists of synthesizing 5α-hydroxy-6β-[2-(1H-imidazol-4-yl)ethylamino]-cholestan-3β-yl ethyl carbonate as follows:

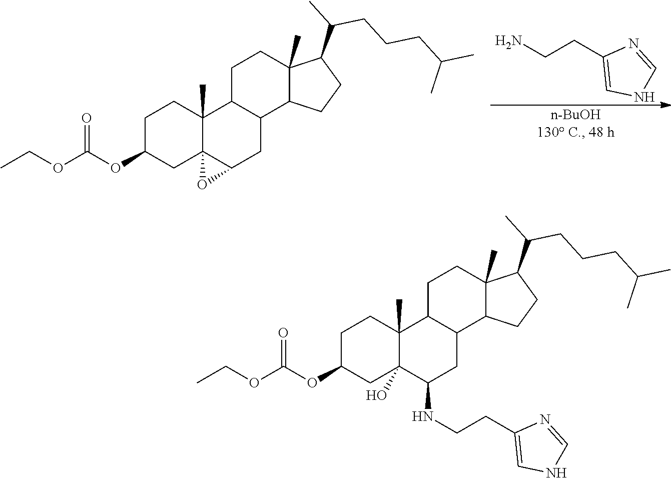

Histamine in its basic form (754.5 mg, 6.8 mmol) is added after complete dissolution of 2.09 g of 5,6α-epoxycholestan-3β-yl ethyl carbonate (at 76%, 3.4 mmol) under 20 ml of butanol. The reaction mixture is stirred under reflux for 48 hours. The progress of the reaction is monitored by thin-layer chromatography (TLC) to monitor the conversion of the 5,6α-epoxycholestan-3β-yl ethyl carbonate. After cooling, the reaction mixture is diluted in 20 mL of methyl tert-butyl ether, and the organic phase is washed 3 times with 20 mL of saturated NaCl solution. The organic phase is dried over anhydrous MgSO₄. The crude reaction product is purified in a silica gel chromatographic column on an automatic purifier. The eluent used is a dichloromethane/ethyl acetate mixture from 100-0% to 0-100%. A white powder of 480 mg of 5α-hydroxy-6β-[2-(1H-imidazol-4-yl)ethylamino]-cholestan-3β-yl ethyl carbonate is obtained, corresponding to 24% yield.

¹H-NMR (500 MHz, MeOD-4d): δ (ppm) 7.54 (s, 1H), 6.80 (s, 1H), 5.00-4.93 (q, 1H), 2.89-2.84 (m, 1H), 2.73-2.62 (m, 3H), 2.36 (s, 1H), 2.08-2.03 (t, 1H), 1.96-1.94 (d, 1H) 1.83-1.79 (m, 2H), 1.66-0.97 (m, 32H), 0.90-0.89 (d, 3H), 0.85-0.84 (dd, 6H), 0.64 (s, 3H).

### Example 6: Preparation of a Dilactate Salt of 5α-hydroxy-6β-[2-(1H-imidazol-4-yl)ethylamino]-cholestan-3β-yl ethyl carbonate (DX121 in the Dilactate Form)

A dilactate salt of the compound 5α-hydroxy-6β-[2-(1H-imidazol-4-yl)ethylamino]-cholestan-3β-yl ethyl carbonate was prepared as follows:

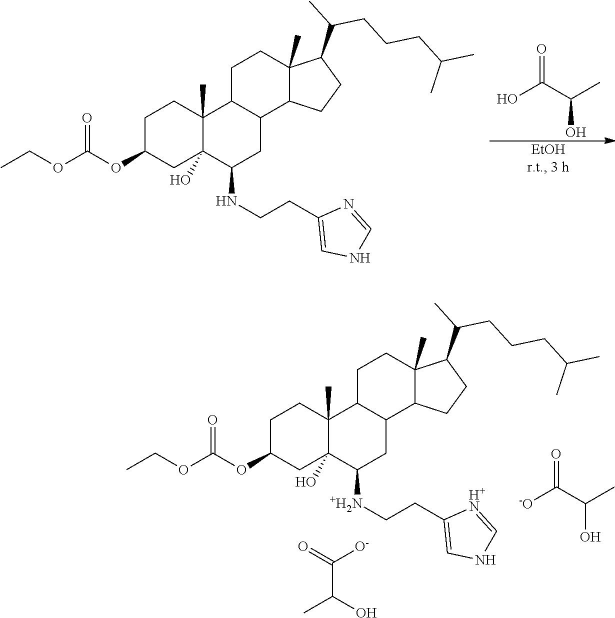

141.6 mg of lactic acid (1.57 mmol) was added to a solution of 460 mg of 5α-hydroxy-6β-[2-(1H-imidazol-4-yl)ethylamino]-cholestan-3β-yl ethyl carbonate (0.785 mmol) in 8 mL of anhydrous ethanol with stirring. Stirring was maintained at room temperature for three hours. Vacuum evaporation of the organic solvent gives a white powder of 1.58 g of 5α-hydroxy-6β-[2-(1H-imidazol-4-yl)ethylamino]-cholestan-3β-yl ethyl carbonate dilactate.

¹H-NMR (500 MHz, MeOD-4d): δ (ppm) 7.57 (s, 1H), 6.76 (s, 1H), 4.74-4.70 (q, 1H), 3.84-3.78 (q, 4H), 3.31-3.27 (m, 2H), 3.13-3.08 (m, 1H), 2.99-2.94 (m, 1H), 2.73-2.67 (m, 3H), 2.04-2.00 (t, 1H), 1.75-1.72 (d, 1H), 1.64-1.39 (m, 8H), 1.28-0.69 (m, 28H), 0.64-0.63 (d, 3H), 0.57-0.56 (d, 6H), 0.45 (s, 3H).

### Example 7: Synthesis of the Compound of Formula (I) 5α-hydroxy-6β-[2-(1H-imidazol-4-yl)ethylamino]cholestan-3β-yl butyl carbonate (Called DX119, in the Basic Form)

The first step consists of synthesizing, starting from the commercial product cholestan-3β-yl butyl carbonate, the compound 5,6α-epoxycholestan-3β-yl butyl carbonate as follows:

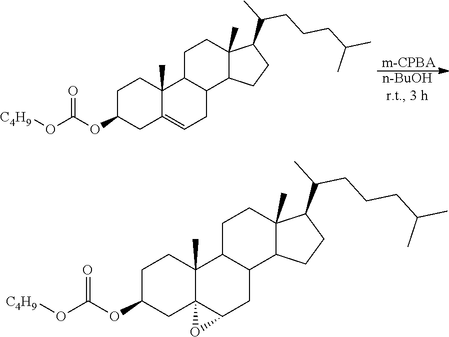

Meta-chloroperoxybenzoic acid (at 77%, 1.28 g, 5.7 mmol) is dissolved in dichloromethane (30 mL) and added dropwise in the space of 30 minutes to a mixture of cholestan-3β-yl butyl carbonate (2.14 g, 4.4 mmol) dissolved in dichloromethane (15 mL). Stirring is maintained at room temperature for three hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 2.21 g of a white powder corresponding to the mixture of the two isomers: 5,6α-epoxycholestan-3β-yl butyl carbonate (77%) and 5,6β-epoxycholestan-3β-yl butyl carbonate (23%). The white powder is used as it is without additional purification.

¹H-NMR (500 MHz, CDCl₃): δ (ppm) 4.86-4.79 (q, 1H), 2.91-2.89 (d, 1H), 2.22-2.18 (t, 1H), 2.09-2.04 (m, 1H), 1.98-1.87 (m, 2H), 1.84-0.93 (m, 33H), 0.89-0.87 (d, 6H), 0.86-0.84 (d, 6H), 0.60 (s, 3H).

The second step consists of synthesizing 5α-hydroxy-6β-[2-(1H-imidazol-4-yl)ethylamino]-cholestan-3β-yl butyl carbonate (DX119 in the basic form) as follows:

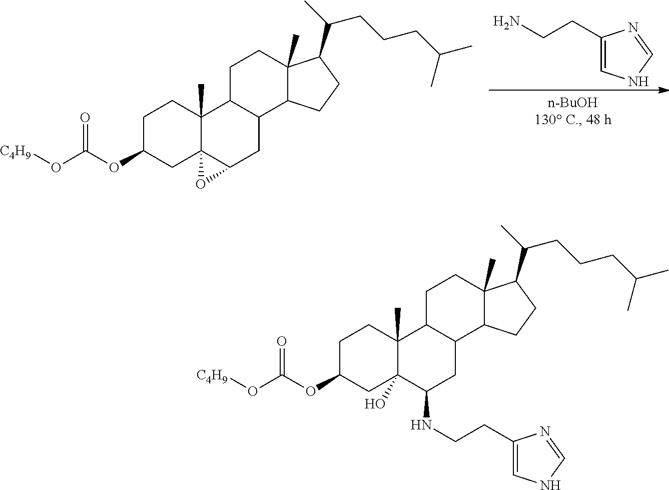

Histamine in its basic form (759.8 mg, 6.8 mmol) is added after complete dissolution of 2.21 g of 5,6α-epoxycholestan-3β-yl butyl carbonate (at 77%, 3.4 mmol) under 20 ml of butanol. The reaction mixture is stirred under reflux for 48 hours. The progress of the reaction is monitored by thin-layer chromatography (TLC) to monitor the conversion of the 5,6α-epoxycholestan-3β-yl butyl carbonate. After cooling, the reaction mixture is diluted in 20 mL of methyl tert-butyl ether, and the organic phase is washed 3 times with 20 mL of saturated NaCl solution. The organic phase is dried over anhydrous MgSO₄. The crude reaction product is purified by column chromatography on an automatic purifier. The eluent used is a dichloromethane/ethyl acetate mixture from 100-0% to 0-100%. A white powder of 814 mg of 5α-hydroxy-6β-[2-(1H-imidazol-4-yl)ethylamino]-cholestan-3β-yl butyl carbonate is obtained, corresponding to 39% yield.

¹H-NMR (500 MHz, MeOD-4d): δ (ppm) 7.54 (s, 1H), 6.80 (s, 1H), 5.00-4.93 (q, 1H), 4.06-4.03 (m, 2H), 2.91-2.87 (m, 1H), 2.73-2.65 (m, 3H), 2.39 (s, 1H), 2.09-2.05 (t, 1H), 1.96-1.93 (d, 1H) 1.84-1.77 (m, 2H), 1.66-0.96 (m, 31H), 0.92-0.88 (m, 6H), 0.85-0.83 (dd, 6H), 0.64 (s, 3H).

### Example 8: Preparation of a Dilactate Salt of 5α-hydroxy-6β-[2-(1H-imidazol-4-yl)ethylamino]-cholestan-3β-yl butyl carbonate (DX119 in the Dilactate Form)

A dilactate salt of the compound 5α-hydroxy-6β-[2-(1H-imidazol-4-yl)ethylamino]-cholestan-3β-yl butyl carbonate was prepared as follows:

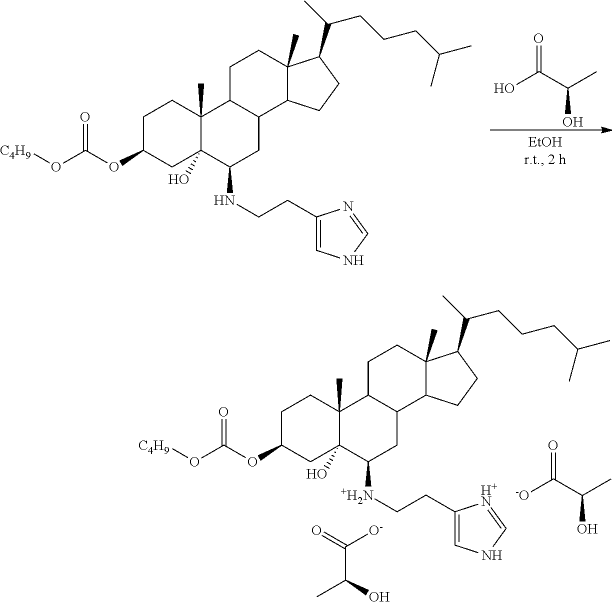

33.7 mg of lactic acid (0.37 mmol) was added to a solution of 114 mg of 5α-hydroxy-6β-[2-(1H-imidazol-4-yl)ethylamino]-cholestan-3β-yl butyl carbonate (0.186 mmol) in 2 mL of anhydrous ethanol with stirring. Stirring was maintained at room temperature for three hours. Vacuum evaporation of the organic solvent gives a white powder of 148 mg of 5α-hydroxy-6β-[2-(1H-imidazol-4-yl)ethylamino]-cholestan-3β-yl butyl carbonate dilactate.

¹H-NMR (500 MHz, MeOD-4d): δ (ppm) 7.70 (s, 1H), 6.97 (s, 1H), 5.02-4.95 (q, 1H), 4.08-4.03 (q, 4H), 3.37-3.35 (m, 1H), 3.26-3.20 (m, 1H), 2.95 (s, 1H), 2.87 (s, 1H), 2.31-2.26 (t, 2H), 2.02-1.99 (d, 1H), 1.92-0.94 (m, 39H), 0.90-0.88 (m, 6H), 0.84-0.82 (dd, 6H), 0.72 (s, 3H).

### Example 9: Synthesis of the Compound of Formula (I) 5α-hydroxy-6β-[2-(1H-imidazol-4-yl)ethylamino]-cholestan-3β-yl-(2-(1H-imidazol-4-yl)ethyl)carbamate (Called DX117, in the Basic Form)

The first step is synthesis of the compound cholestan-3β-yl phenyl carbonate comprising the following steps:

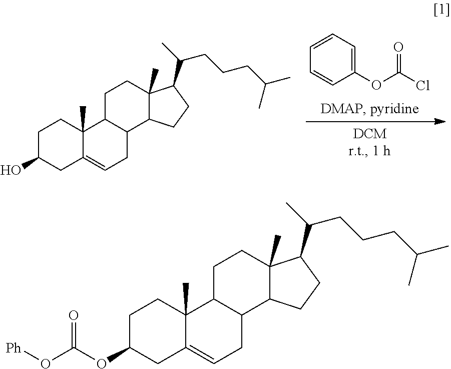

In a 100 mL flask with ground neck, 20 mL of dichloromethane is added to dissolve 7.58 g of cholesterol (19.6 mmol) and 1.0 g of 4-dimethylaminopyridine (DMAP, 8.2 mmol). Then 15 mL of anhydrous pyridine (185.5 mmol) and 3.4 ml of phenyl chloroformate (24.1 mmol) are added and the whole is stirred at room temperature for 1 hour. At the end of the hour, the reaction mixture is diluted by adding 35 mL of dichloromethane and the reaction mixture is washed three times with 70 mL of an aqueous solution of HCl (1M). The organic phase is dried over anhydrous MgSO₄. Vacuum evaporation of the organic solvent gives 8.79 g of a white powder corresponding to the desired product, corresponding to 98% yield.

¹H-NMR (500 MHz, CDCl₃): δ (ppm) 7.31-7.28 (m, 2H), 7.17-7.13 (m, 1H), 7.12-7.10 (d, 2H), 5.35-5.34 (q, 1H), 4.54-4.47 (m, 1H), 2.45-2.36 (m, 2H), 1.98-1.61 (m, 6H), 1.54-0.88 (m, 23H), 0.85-0.84 (d, 3H), 0.80-0.78 (d, 6H), 0.61 (s, 3H).

The second step consists of synthesizing, starting from cholestan-3β-yl phenyl carbonate, the compound 5,6α-epoxycholestan-3β-yl phenyl carbonate as follows:

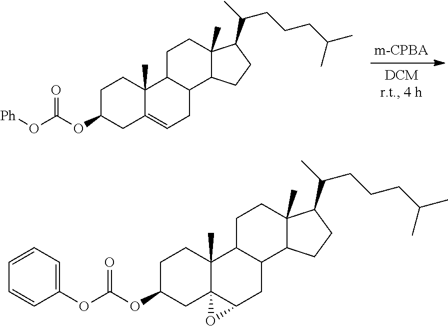 [SMILES: CC(C)CCCC(C)[C@H]1CCC2C3CC=C4C[C@@H](OC(=O)Oc5ccccc5)CC[C@]4(C)C3CC[C@@]21C ; CC(C)CCCC(C)[C@H]1CCC2C3C[C@@H]4O[C@@]45C[C@@H](OC(=O)Oc4ccccc4)CC[C@]5(C)C3CC[C@@]21C]

Meta-chloroperoxybenzoic acid (at 77%, 5.08 g, 22.7 mmol) is dissolved in dichloromethane (70 mL) and added dropwise in the space of 1 h to a mixture of cholestan-3β-yl phenyl carbonate (8.79 g, 17.3 mmol) dissolved in dichloromethane (70 mL). Stirring is maintained at room temperature for three hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 9.04 g of clear oil corresponding to the mixture of the two isomers: 5,6α-epoxycholestan-3β-yl phenyl carbonate and 5,6β-epoxycholestan-3β-yl phenyl carbonate. The mixture is redissolved in 10 mL of Et₂O and 40 mL of EtOH is added to obtain a white precipitate. The solution is filtered and the precipitate is washed with EtOH. The procedure gives 7.23 g of a white powder rich in 5,6α-epoxy-cholestan-3β-yl phenyl carbonate corresponding to 88% of 89% yield (76% enantiomeric excess).

¹H-NMR (500 MHz, CDCl₃): δ (ppm) 7.38-7.35 (m, 2H), 7.24-7.21 (m, 1H), 7.18-7.16 (d, 2H), 4.96-4.90 (q, 1H), 2.93 (s, 1H), 2.33-2.29 (t, 1H), 2.18-2.15 (d, 1H), 1.97-1.90 (m, 2H), 1.84-1.74 (m, 3H), 1.58-0.95 (m, 24H), 0.90-0.88 (d, 3H), 0.87-0.85 (dd, 6H), 0.61 (s, 3H).

The third step consists of synthesizing 5α-hydroxy-6β-[2-(1H-imidazol-4-yl)ethylamino]-cholestan-3β-yl-(2-(1H-imidazol-4-yl)ethyl)carbamate (DX117 in the basic form) as follows:

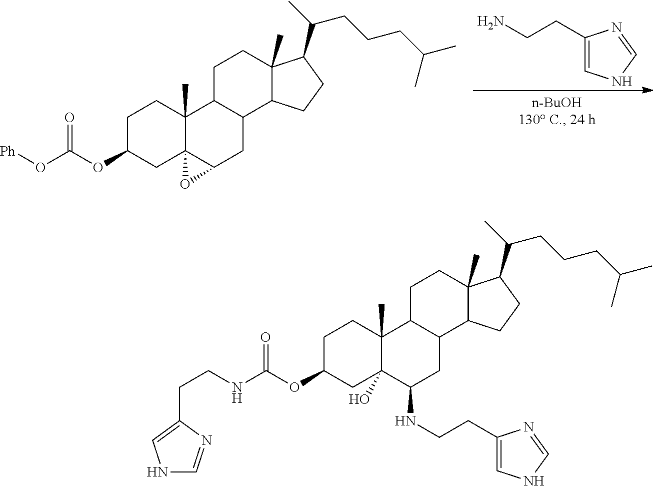 [SMILES: CC(C)CCCC(C)[C@H]1CCC2C3C[C@@H]4O[C@@]45C[C@@H](OC(=O)Oc4ccccc4)CC[C@]5(C)C3CC[C@@]21C ; NCCc1c[nH]cn1 ; CC(C)CCCC(C)[C@H]1CCC2C3C[C@@H](NCCc4c[nH]cn4)[C@@]4(O)C[C@@H](OC(=O)NCCc5c[nH]cn5)CC[C@]4(C)C3CC[C@@]21C]

Histamine in its basic form (1.13 g, 10.2 mmol) is added after complete dissolution of 1.0 g of 5,6α-epoxy-cholestan-3β-yl phenyl carbonate (at 88%, 1.7 mmol) with 30 mL of butanol. The reaction mixture is stirred under reflux for 48 hours. The progress of the reaction is monitored by thin-layer chromatography (TLC) to monitor the conversion of the 5,6α-epoxycholestan-3β-yl phenyl carbonate. The mixture is transferred to a separatory funnel and the organic products are extracted twice with 15 mL of methyl tert-butyl ether and twice more with 15 mL of ethyl acetate. The organic phases are combined, and dried on anhydrous MgSO₄. The crude reaction product is purified in a silica gel chromatographic column on an automatic purifier. The eluent used is a mixture of ethyl acetate-MeOH from 95-5% to 80-20% and finally DCM-MeOH—NH₄OH 75-20-5%. A white powder of 530 mg of 5α-hydroxy-6β-[2-(1H-imidazol-4-yl)ethylamino]-cholestan-3β-yl-(2-(1H-imidazol-4-yl)ethyl)carbamate is obtained, corresponding to 48% yield.

¹H-NMR (500 MHz, MeOD-4d): δ (ppm) 7.60 (s, 1H), 7.58 (s, 1H), 6.86 (s, 1H), 6.83 (s, 1H), 5.02-4.95 (q, 1H), 3.31-3.30 (m, 2H), 2.99-2.97 (m, 1H), 2.77-2.74 (m, 5H), 2.45 (s, 1H), 2.09-2.05 (t, 1H), 2.00-1.98 (d, 1H), 1.86-1.81 (m, 2H), 1.69-1.00 (m, 27H), 0.93-0.92 (d, 3H), 0.89-0.87 (d, 6H), 0.69 (s, 3H).

### Example 10: Preparation of a Trilactate Salt of 5α-hydroxy-6β-[2-(1H-imidazol-4-yl)ethylamino]-cholestan-3β-yl-(2-(1H-imidazol-4-yl)ethyl)carbamate (DX117 in the Trilactate Form)

A trilactate salt of the compound 5α-hydroxy-6β-[2-(1H-imidazol-4-yl)ethylamino]-cholestan-3β-yl-(2-(1H-imidazol-4-yl)ethyl)carbamate was prepared as follows:

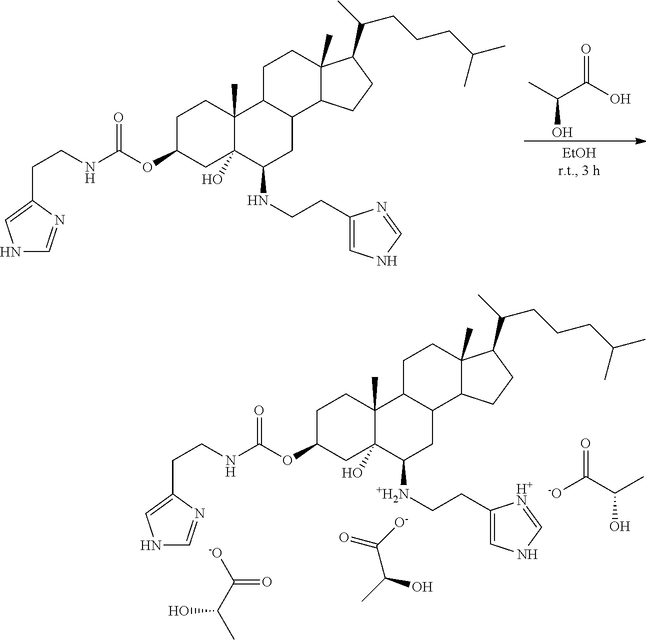

103.4 mg of lactic acid (1.15 mmol) was added to a solution of 251.2 mg of 5α-hydroxy-6β-[2-(1H-imidazol-4-yl)ethylamino]-cholestan-3β-yl-(2-(1H-imidazol-4-yl)ethyl) carbamate (0.39 mmol) in 5 mL of anhydrous ethanol with stirring. Stirring was maintained at room temperature for three hours. Vacuum evaporation of the organic solvent gives a white powder of 1.58 g of 5α-hydroxy-6β-[2-(1H-imidazol-4-yl)ethylamino]-cholestan-3β-yl-(2-(1H-imidazol-4-yl)ethyl)carbamate trilactate.

¹H-NMR (500 MHz, MeOD-4d): δ (ppm) 8.20 (s, 1H), 7.60 (s, 1H), 6.96 (s, 1H), 6.85 (s, 1H), 4.85-4.78 (m, 1H), 3.95-3.91 (q, 4H), 3.43-3.39 (q, 1H), 3.25-3.07 (m, 5H), 2.84-2.83 (m, 2H), 2.74 (s, 1H), 2.68-2.65 (m, 2H), 2.11-2.07 (t, 1H), 1.86-1.84 (d, 1H), 1.69-0.81 (m, 35H), 0.75-0.74 (d, 3H), 0.69-0.67 (d, 6H), 0.57 (s, 3H).

### Example 11: Synthesis of the Compound of Formula (I) 5α-hydroxy-6β-[2-(1H-imidazol-4-yl)ethylamino]cholestan-3β-yl-diethyl carbamate (Called DX131, in the Basic Form)

The first step is synthesis of the compound cholestan-3β-diethyl carbamate comprising the following steps:

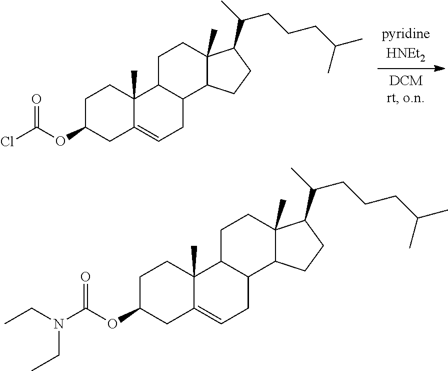

In a 100 mL flask with ground neck, 2.0 mL of anhydrous pyridine (d=0.978 g/mL; 24.7 mmol) is added to a mixture of cholesterol chloroformate (9.07 g, 20.2 mmol) dissolved in 40 mL of dichloromethane. 5.0 mL ethylamine (d=1.248 g/mL; 48.3 mmol) is added and the whole is stirred at room temperature overnight. The reaction mixture is diluted with 60 mL of dichloromethane and washed 5 times with 100 mL of an aqueous solution of HCl 3.7 vol %. The organic phase is dried over anhydrous MgSO₄ and the organic solvent is evaporated under vacuum. The white solid was dissolved with 100 mL of Et₂O and precipitated with 100 mL of MeOH, then the solution is filtered and the precipitate is washed with cold MeOH. The procedure gives 9.30 g of a white powder corresponding to cholestan-3β-diethyl carbamate (yield of 95%).

¹H-NMR (500 MHz, CDCl₃): δ (ppm) 5.37-5.36 (d, 1H), 4.54-4.49 (m, 1H), 2.38-2.37 (dd, 1H), 2.31-2.27 (t, 1H), 2.02-1.80 (m, 5H), 1.60-0.93 (m, 34H), 0.92-0.91 (d, 3H), 0.87-0.86 (d, 6H), 0.67 (s, 3H).

The second step consists of synthesizing, starting from cholestan-3β-diethyl carbamate, the compound 5,6α-epoxycholestan-3β-diethyl carbamate as follows:

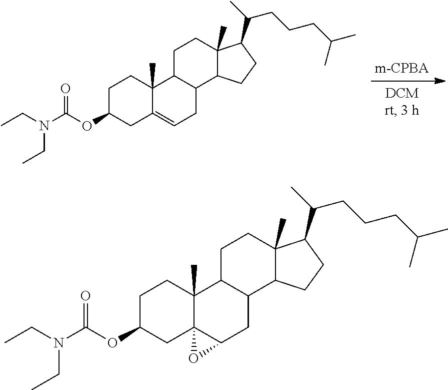

Meta-chloroperoxybenzoic acid (at 77%, 4.47 g, 19.9 mmol) is dissolved in dichloromethane (100 mL) and added dropwise in the space of 1 h to a mixture of cholestan-3β-diethyl carbamate (7.45 g, 15.3 mmol) dissolved in dichloromethane (50 mL). Stirring is maintained at room temperature for three hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 7.81 g of a white solid corresponding to the mixture of the two isomers: 5,6α-epoxycholestan-3β-diethyl carbamate (69%) carbamate and 5,6β-epoxycholestan-3β-diethyl carbamate (31%). The white solid is dissolved in 50 mL of dichloromethane and 100 mL of MeOH is added to precipitate 4.27 g of a white powder rich in 5,6α-epoxycholestan-3β-diethyl carbamate (86%).

¹H-NMR (500 MHz, CDCl₃): δ (ppm) 4.88-4.83 (q, 1H), 2.88 (s, 1H), 2.16-2.13 (t, 1H) 2.05-2.03 (d, 1H), 1.97-0.93 (m, 39H), 0.89-0.88 (d, 3H), 0.86-0.85 (d, 6H), 0.60 (s, 3H).

The third step consists of synthesizing 5α-hydroxy-6β-[2-(1H-imidazol-4-yl)ethylamino]cholestane-3β-diethyl carbamate (DX131 in the basic form) as follows:

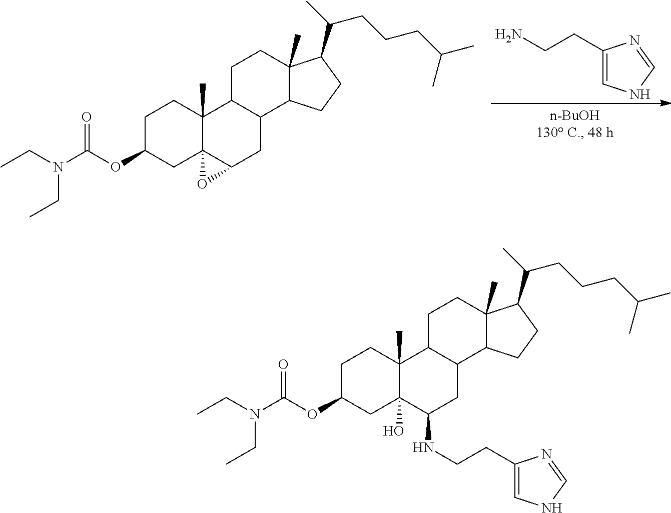

Histamine in its basic form (756.3 mg, 6.8 mmol) is added after complete dissolution of 2.00 g of 5,6α-epoxycholestan-3β-diethyl carbamate (at 86%, 3.4 mmol) with 7 ml of butanol. The reaction mixture is stirred under reflux for 48 hours. The progress of the reaction is monitored by thin-layer chromatography (TLC) to monitor the conversion of the 5,6α-epoxycholestan-3β-diethyl carbamate. After cooling, the reaction mixture is diluted in 7 mL of methyl tert-butyl ether, the organic phase is washed 3 times with 7 mL of saturated NaCl solution. The organic phase is dried over anhydrous MgSO₄. The crude reaction product is purified in a silica gel chromatographic column. The eluent used is a hexane-ethyl acetate mixture from 90-10% to 0-100%, and then ethyl acetate-methanol from 90-10% to 70-30%. A white powder of 0.45 g of 5α-hydroxy-6β-[2-(1H-imidazol-4-yl)ethylamino]cholestan-3β-yl-diethyl carbamate is obtained, corresponding to 22% yield.

¹H-NMR (500 MHz, MeOD-4d): δ (ppm) 7.61 (s, 1H), 6.89 (s, 1H), 5.07-5.01 (q, 1H), 3.05-2.99 (m, 1H), 2.82-2.79 (m, 3H), 2.49 (s, 1H), 2.19-2.14 (t, 1H), 2.02-1.99 (bd, 1H), 1.88-1.82 (m, 2H), 1.73-0.96 (m, 37H), 0.95-0.93 (d, 3H), 0.90-0.88 (dd, 6H), 0.70 (s, 3H).

### Example 12: Preparation of a Dilactate Salt of 5α-hydroxy-6β-[2-(1H-imidazol-4-yl)ethylamino]cholestan-3β-yl-diethyl carbamate (DX131 in the Dilactate Form)

A dilactate salt of the compound 5α-hydroxy-6β-[2-(1H-imidazol-4-yl)ethylamino]cholestan-3β-yl-diethyl carbamate was prepared as follows:

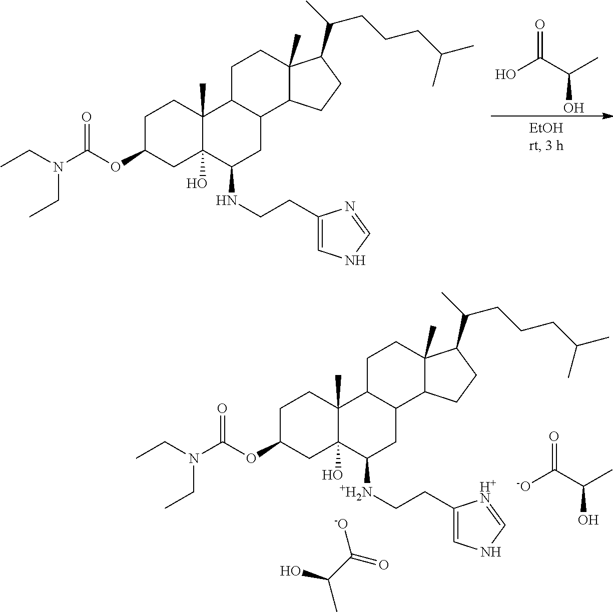

132.7 mg of lactic acid (1.34 mmol) was added to a solution of 449.1 mg of 5α-hydroxy-6β-[2-(1H-imidazol-4-yl)ethylamino]cholestan-3β-yl-diethyl carbamate (0.73 mmol) in 7.5 mL of anhydrous ethanol with stirring. Stirring was maintained at room temperature for three hours. Vacuum evaporation of the organic solvent gives a white powder of 580.6 g of 5α-hydroxy-6β-[2-(1H-imidazol-4-yl)ethylamino]cholestan-3β-yl-diethyl carbamate dilactate.

¹H-NMR (500 MHz, MeOD-4d): δ (ppm) 7.66 (s, 1H), 6.94 (s, 1H), 5.01-4.95 (q, 1H), 4.06-4.01 (q, 2H), 3.53-3.49 (m, 1H), 3.37-3.32 (q, 1H), 3.22-3.17 (m, 6H), 2.94-2.91 (t, 1H), 2.86 (bd, 1H), 2.29-2.25 (t, 1H) 1.98-1.95 (d, 1H), 1.84-0.92 (m, 40H), 0.86-0.85 (d, 3H), 0.80-0.78 (d, 6H), 0.68 (s, 3H).

### Example 13: Synthesis of the Compound of Formula (I) 5α-hydroxy-6β-[2-(1H-imidazol-4-yl)ethylamino]cholestan-3β-yl propionyltyrosine (Called DX133, in the Basic Form)

The first step is the synthesis of N-propionyltyrosine starting from the amino acid tyrosine:

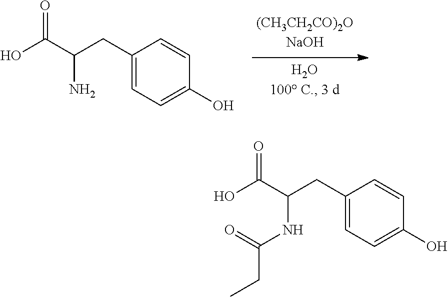 [SMILES: NC(Cc1ccc(O)cc1)C(=O)O ; CCC(=O)NC(Cc1ccc(O)cc1)C(=O)O]

10 mL of propionic anhydride (d=1.01 g/mL; 77.7 mmol) was added after complete dissolution of 2.33 g of NaOH (58.3 mmol) and 3.52 g of tyrosine (62.1 mmol) in 60 mL of deionized water at 100° C. ambient. The reaction mixture is stirred under reflux for 3 days. At the end of this time, the reaction was neutralized by adding HCl until pH=6 and transferred to a separatory funnel and extracted three times with ethyl acetate. The organic phases thus obtained were combined and dried over MgSO₄ and then evaporated, giving a clear oil corresponding to the mixture of N-propionyltyrosine and the unreacted propionic anhydride. The oil was dissolved with EtOH and dried under vacuum to give a white powder of 3.81 g (70% yield).

¹H-NMR (500 MHz, MeOD-4d): δ (ppm) 7.03-7.01 (d, 2H), 6.69-6.67 (d, 2H), 4.59-4.56 (m, 1H), 3.11-3.07 (m, 1H), 2.86-2.81 (m, 1H), 2.19-2.14 (q, 2H), 1.04-1.01 (t, 3H).

The second step is the esterification reaction between cholesterol and N-propionyltyrosine to obtain the compound cholestan-3β-yl propionyltyrosine

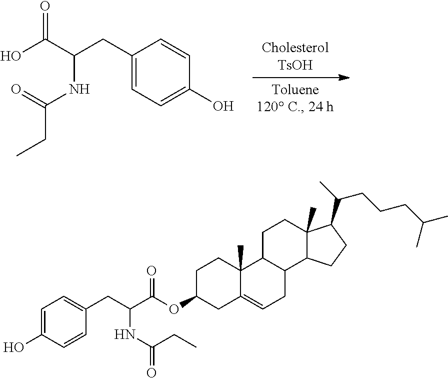 [SMILES: CCC(=O)NC(Cc1ccc(O)cc1)C(=O)O ; CCC(=O)NC(Cc1ccc(O)cc1)C(=O)O[C@H]1CC[C@@]2(C)C(=CCC3C2CC[C@@]2(C)C3CC[C@@H]2C(C)CCCC(C)C)C1]

3.81 g of N-propionyltyrosine (13.6 mmol), 5.26 g of cholesterol (13.5 mmol) and 480 mg of paratoluenesulfonic monohydrate acid (TsOH, 1.39 mmol) were transferred to a 100 mL flask with ground neck and dissolved in 20 mL of toluene. TsOH was added in two portions; the second portion was added 6 hours after the first. The reaction mixture is stirred under reflux for 24 hours. At the end of this day of reaction, the reaction was neutralized by adding NaOH until pH=12. The mixture is transferred to a separatory funnel and extracted three times with ethyl acetate. The organic phases thus obtained were combined and dried over MgSO₄ and then evaporated, giving a brownish-white solid. The crude reaction product is purified in a silica gel chromatographic column. The eluent used is a mixture of hexane/ethyl acetate 90-10% to 30-70%. A brownish-white solid of 7.83 g of cholestan-3β-yl propionyltyrosine is obtained, corresponding to 96% yield.

¹H-NMR (500 MHz, CDCl₃): δ (ppm) 6.97-6.95 (dd, 2H), 6.73-6.70 (dd, 2H), 6.41 (bs, 1H), 5.98-5.97 (d, 1H), 5.38-5.36 (t, 1H), 4.84-4.80 (m, 1H), 4.66-4.60 (m, 1H), 3.09-2.96 (m, 2H), 2.36-2.19 (m, 4H), 2.02-1.95 (m, 2H), 1.88-1.79 (m, 3H), 1.71-0.93 (m, 27H), 0.92-0.91 (d, 3H), 0.87-0.85 (d, 6H), 0.68 (s, 3H).

The third step consists of synthesizing, starting from cholestan-3β-yl propionyltyrosine, the compound 5,6α-epoxycholestan-3β-yl propionyltyrosine as follows:

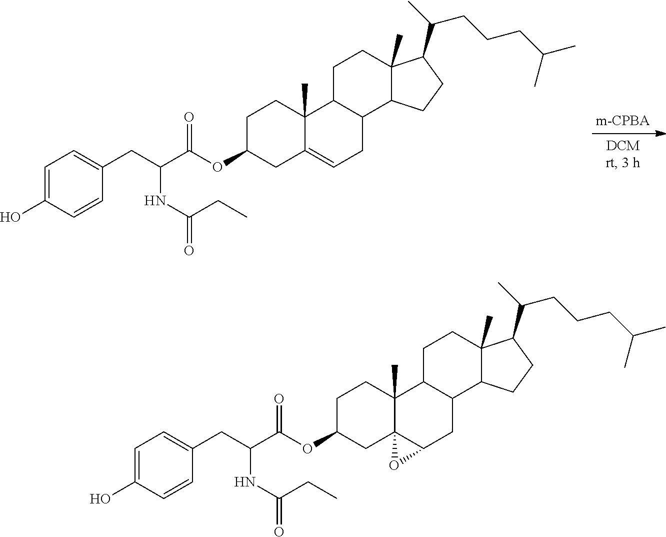 [SMILES: CCC(=O)NC(Cc1ccc(O)cc1)C(=O)O[C@H]1CC[C@@]2(C)C(=CCC3C2CC[C@@]2(C)C3CC[C@@H]2C(C)CCCC(C)C)C1 ; CCC(=O)NC(Cc1ccc(O)cc1)C(=O)O[C@H]1CC[C@]2(C)C3CC[C@@]4(C)C(CC[C@@H]4C(C)CCCC(C)C)C3C[C@@H]3O[C@@]32C1]

Meta-chloroperoxybenzoic acid (at 77%, 1.79 g, 8.0 mmol) is dissolved in dichloromethane (45 mL) and added dropwise in the space of 30 minutes to a mixture of cholestan-3β-yl propionyltyrosine (4.66 g, 7.7 mmol) dissolved in dichloromethane (20 mL). Stirring is maintained at room temperature for three hours. The reaction mixture is washed twice with an aqueous solution of Na₂S₂O₃ (10 wt %), twice with a saturated solution of NaHCO₃ and once with saturated NaCl solution. The organic phase is dried over anhydrous MgSO₄. Vacuum evaporation of the organic solvent gives 6.29 g of an oil corresponding to the mixture of the two isomers: 5,6α-epoxycholestan-3β-yl propionylglycine and 5,6β-epoxycholestan-3β-yl propionylglycine. The mixture is redissolved in 20 mL of dichloromethane and 80 mL of EtOH is added, obtaining a brownish-white precipitate. The brownish-white precipitate obtained was filtered and washed with MeOH. During washing with MeOH, the white powder passes through the filter; the resultant filtrate was dried under vacuum, giving 4.33 g of a white powder rich in 5,6α-epoxycholestan-3β-yl propionyltyrosine corresponding to 90% yield (36% enantiomeric excess).

¹H-NMR (500 MHz, CDCl₃): δ (ppm) 6.95-6.93 (dd, 2H), 6.73-6.71 (dd, 2H), 6.48 (bs, 1H), 4.99-4.92 (m, 1H), 4.80-4.75 (m, 1H), 3.07-2.89 (m, 4H), 2.22-0.93 (m, 36H), 0.89-0.88 (d, 3H), 0.86-0.85 (dd, 6H), 0.61 (s, 3H).

The fourth step consists of synthesizing 5α-hydroxy-6β-[2-(1H-imidazol-4-yl)ethylamino]cholestan-3β-yl propionyltyrosine as follows (DX133 in the basic form):

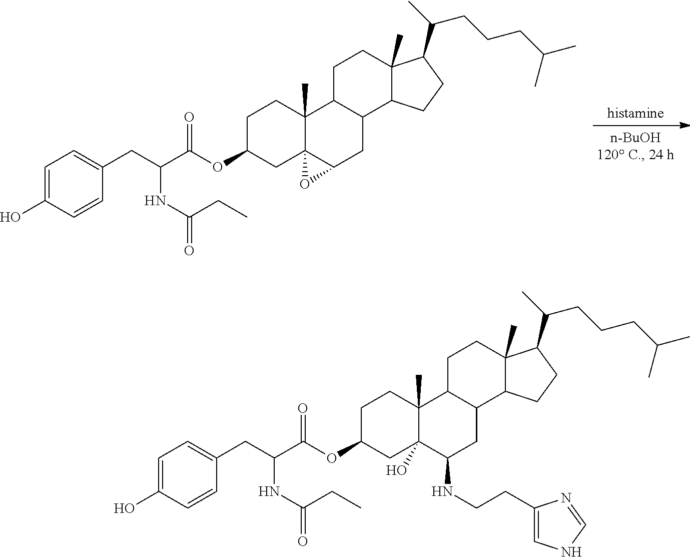 [SMILES: CCC(=O)NC(Cc1ccc(O)cc1)C(=O)O[C@H]1CC[C@]2(C)C3CC[C@@]4(C)C(CC[C@@H]4C(C)CCCC(C)C)C3C[C@@H]3O[C@@]32C1 ; CCC(=O)NC(Cc1ccc(O)cc1)C(=O)O[C@H]1CC[C@]2(C)C3CC[C@@]4(C)C(CC[C@@H]4C(C)CCCC(C)C)C3C[C@@H](NCCc3c[nH]cn3)[C@@]2(O)C1]

Histamine in its basic form (449 mg, 4.0 mmol) is added after complete dissolution of 1.0 g of 5,6α-epoxycholestan-3β-yl propionyltyrosine (at 63%, 1.0 mmol) under 5 ml of butanol. The reaction mixture is stirred under reflux for 24 hours. The progress of the reaction is monitored by thin-layer chromatography (TLC) to monitor the conversion of the 5,6α-epoxycholestan-3β-yl propionyltyrosine. After cooling, the reaction mixture is diluted in 5 mL of methyl tert-butyl ether, the organic phase is washed twice with 5 mL of saturated NaCl solution and once with 5 mL of a saturated solution of NaHCO₃. The organic phase is dried over anhydrous MgSO₄. The crude reaction product is purified in a silica gel chromatographic column. The eluent used is an ethyl acetate-methanol mixture 100-0% to 70-30%. A white powder of 220 mg of 5α-hydroxy-6β-[2-(1H-imidazol-4-yl)ethylamino]cholestan-3β-yl propionyltyrosine is obtained, corresponding to 28% yield.

¹H-NMR (500 MHz, MeOD-4d): δ (ppm) 7.60 (s, 1H), 7.02-7.00 (d, 2H), 6.86 (s, 1H), 6.70-6.69 (d, 2H), 5.20-5.13 (q, 1H), 4.55-4.52 (q, 1H), 3.02-2.72 (m, 6H), 2.41 (s, 1H), 2.21-2.16 (q, 2H), 2.08-2.04 (t, 1H), 2.00-1.97 (d, 1H), 1.88-1.80 (m, 2H), 1.68-1.00 (m, 30H), 0.93-0.91 (d, 3H), 0.89-0.87 (dd, 6H), 0.67 (s, 3H).

### Example 14: Preparation of a Dilactate Salt of 5α-hydroxy-6β-[2-(1H-imidazol-4-yl)ethylamino]cholestan-3β-yl propionyltyrosine (DX133 in the Lactate Form)

A dilactate salt of the compound 5α-hydroxy-6β-[2-(1H-imidazol-4-yl)ethylamino]cholestan-3β-yl propionyltyrosine was prepared as follows:

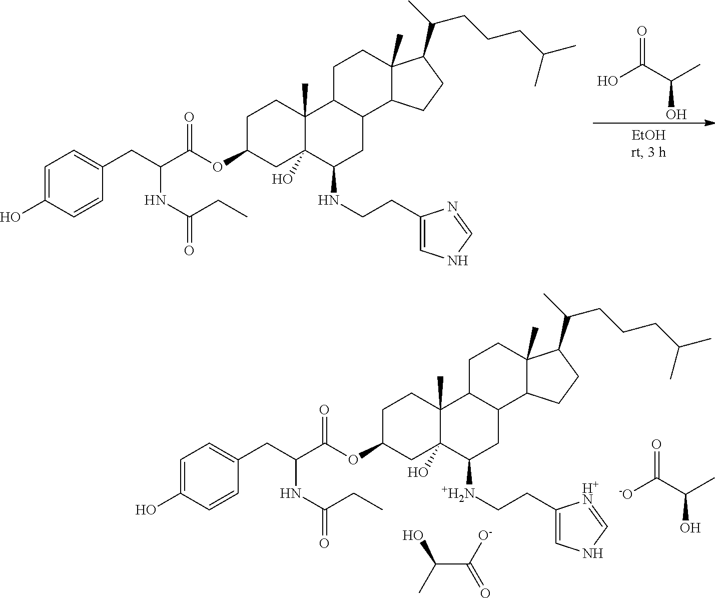

53.1 mg of lactic acid (0.59 mmol) was added to a solution of 212.4 mg of 5α-hydroxy-6β-[2-(1H-imidazol-4-yl)ethylamino]cholestan-3β-yl propionyltyrosine (0.29 mmol) in 3 mL of anhydrous ethanol with stirring. Stirring was maintained at room temperature for three hours. Vacuum evaporation of the organic solvent gives a white powder of 265.5 mg of 5α-hydroxy-6β-[2-(1H-imidazol-4-yl)ethylamino]cholestan-3β-yl propionyltyrosine dilactate.

¹H-NMR (500 MHz, MeOD-4d): δ (ppm) 7.74 (s, 1H), 7.01-6.97 (m, 3H), 6.70-6.64 (dd, 2H), 5.20-5.11 (q, 1H), 4.52-4.44 (m, 1H), 4.13-4.09 (q, 2H), 3.43-3.37 (m, 1H), 3.25-3.21 (m, 1H), 2.98-2.81 (m, 5H), 2.26-2.15 (m, 3H), 2.06-2.03 (bd, 1H), 1.88-1.00 (m, 38H), 0.94-0.93 (d, 3H), 0.88-0.87 (dd, 6H), 0.76 (s, 3H).

The successive reaction pathways are the same steps that were developed for the synthesis of Dendrogenin A, disclosed in particular in patent EP2782923B1, “Method for preparing sterol derivatives”, the content of which is included in the present invention by reference.

Example 15: Synthesis of the compounds (N-propionyl)-L-histidine 5α-hydroxy-6β-[2-(1H-imidazol-4-yl)-ethylamino]-cholestan-3β-yl ester, (N-propionyl)-L-isoleucine 5α-hydroxy-6β-[2-(1H-imidazol-4-yl)-ethylamino]-cholestan-3β-yl ester, (N-propionyl)-L-leucine 5α-hydroxy-6β-[2-(1H-imidazol-4-yl)-ethylamino]-cholestan-3β-yl ester, (N-propionyl)-L-lysine 5α-hydroxy-6β-[2-(1H-imidazol-4-yl)-ethylamino]-cholestan-3β-yl ester, (N-propionyl)-L-phenylalanine 5α-hydroxy-6β-[2-(1H-imidazol-4-yl)-ethylamino]-cholestan-3β-yl ester, (N-propionyl)-L-proline 5α-hydroxy-6β-[2-(1H-imidazol-4-yl)-ethylamino]-cholestan-3β-yl ester, (N-propionyl)-L-tryptophan 5α-hydroxy-6β-[2-(1H-imidazol-4-yl)-ethylamino]-cholestan-3β-yl ester, (N-propionyl)-L-tyrosine 5α-hydroxy-6β-[2-(1H-imidazol-4-yl)-ethylamino]-cholestan-3β-yl ester, (N-propionyl)-L-valine 5α-hydroxy-6β-[2-(1H-imidazol-4-yl)-ethylamino]-cholestan-3β-yl ester, comprises the following steps:
dissolution of the various amino acids in CH₃CN and addition of the reagents DMAP and (BOC)₂O to protect their amino groups,
esterification of the N-propionate amino acids with cholesterol by Fischer reaction catalyzed by TsOH in toluene
The successive reaction pathways are the same steps that were developed for the synthesis of Dendrogenin A (see above).

### Example 16: Pharmacokinetic Study of DX107

The following study is determination by LC/MS in plasma of the various molecules over 3 days (11 measurement points at the end). The graphs are always presented in comparison with DX101, which is the reference.

Protocol

| | Group 1 | Group 2 |
|---|---|---|
| Administration of | DX101 | DX107 |
| the compound | | |
| Dose | 50 mg/kg | 50 mg/kg |
| Route of | oral | oral |
| application | | |
| Animals | rat | rat |
| Size of group | 3 | 3 |
| Samples | plasma | plasma |
| Assay | DX101 | DX107 and DX101 |

Plasma samples taken at 0 (without injection), 5, 10, 15, 30 min, 1 h 4 h 8 h 24 h 48 h 72 h (11 points)

The pharmacokinetic profile of DX107 compared to DX101 is given in FIG. 1. The results are as follows:

| | | | DX101 starting |
|---|---|---|---|
| | DX101 | DX107 | from DX107 |
| Area under curve | 143297 | 89733 | 159259 |
| Concentration max (nM) | 140 | 142 | 178 |
| Time to Cmax (min) | 480 | 60 | 240 |

Conclusions: The results show that the profile of DX107 has quicker absorption in the body and better bioavailability than DX101. It is clearly demonstrated that DX101 is a prodrug, since the presence of DX101 in the body is increased, making it possible to increase the efficacy of the treatment.

### Example 17: Pharmacokinetic Study of DX117

The following study is a determination by LC/MS in plasma of the various molecules over 3 days (11 measurement points at the end). The graphs are always presented in comparison with DX101, which is the reference.

Protocol

| | Group 1 | Group 2 |
|---|---|---|
| Administration of | DX101 | DX117 |
| the compound | | |
| Dose | 50 mg/kg | 50 mg/kg |
| Route of | oral | and |
| application | | |
| Animals | rat | rat |
| Size of group | 3 | 3 |
| Samples | plasma | plasma |
| Assay | DX101 | DX117 and DX101 |

Plasma samples taken at 0 (without injection), 5, 10, 15, 30 min, 1 h 4 h 8 h 24 h 48 h 72 h (11 points)

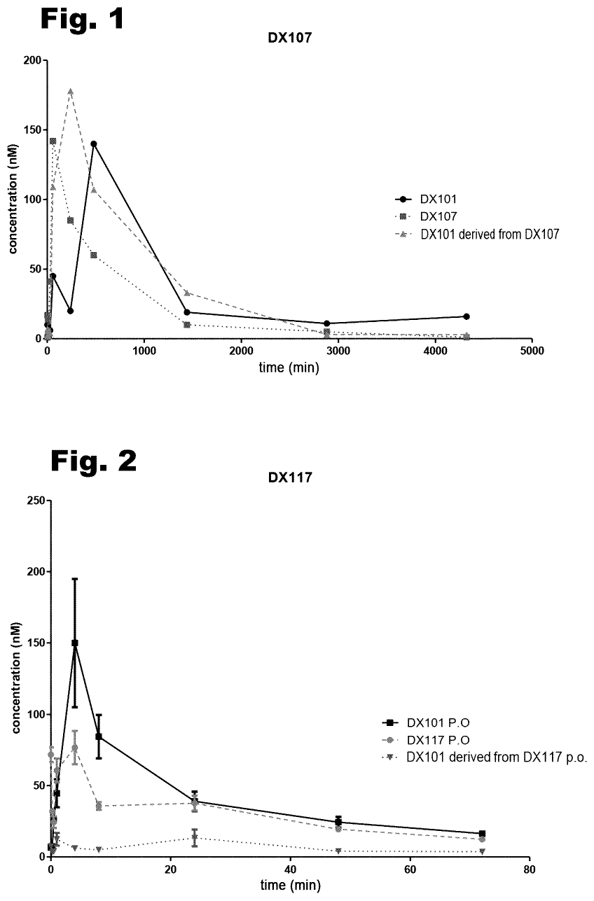

The pharmacokinetic profile of DX117 compared to DX101 is given in FIG. 2. The results are as follows:

| | | | DX101 starting |
|---|---|---|---|
| | DX101 | DX117 | from DX117 |
| Area under curve | 3019 | 2123 | 502.5 |
| Concentration max (nM) | 150 | 76.67 | 13.33 |
| Time to Cmax (min) | 4 | 4 | 24 |

Conclusions: The results clearly show that DX117, and hence the carbamate derivatives, is a prodrug, since the presence of DX101 in the body is confirmed.

### Example 18: Pharmacokinetic Study of DX121

The following study is a determination by LC/MS in plasma of the various molecules over 3 days (11 measurement points at the end). The graphs are always presented in comparison with DX101, which is the reference.

Protocol

| | Group 1 | Group 2 |
|---|---|---|
| Administration of | DX101 | DX121 |
| the compound | | |
| Dose | 50 mg/kg | 50 mg/kg |
| Route of | oral | oral |
| application | | |
| Animals | rat | rat |
| Size of group | 3 | 3 |
| Samples | plasma | plasma |
| Assay | DX101 | DX121 and DX101 |

Plasma samples taken at 0 (without injection), 5, 10, 15, 30 min, 1 h 4 h 8 h 24 h 48 h 72 h (11 points)

The pharmacokinetic profile of DX121 compared to DX101 is given in FIG. 3. The results are as follows:

| | | | DX101 starting |
|---|---|---|---|
| | DX101 | DX121 | from DX121 |
| Area under curve | 3019 | 1000 | 1590 |
| Concentration max (nM) | 150 | 96.33 | 91.33 |
| Time to Cmax (min) | 4 | 1 | 4 |

Conclusions: The results clearly show that DX121, and hence the carbonate derivatives, is a prodrug, since the presence of DX101 in the body is confirmed.

### Example 19: Study of the cytotoxicity of the compounds 5α-hydroxy-6-[2-(1H-imidazol-4-yl)-ethylamino]-cholestan-3β-yl propionate or DX107

For this experiment, a cell culture medium was prepared. The culture medium consists of a Dulbecco's Modified Eagle Medium (DMEM) marketed by Westburg under the reference LO BE12-604F), comprising 4.5 g/L glucose with L-glutamine, to which 10% of fetal calf serum (FCS) is added. Neuro2a cells (murine neuroblastoma) were introduced into this culture medium.

24-well plates were seeded with 10000 Neuro2a cells per well. After culture for 72 hours (h) in normal conditions, i.e. in an incubator at a temperature of 37° C. at 5% CO₂, the Neuro2a cells were treated for 48 h with 5α-hydroxy-6β-[2-(1H-imidazol-4-yl)-ethylamino]-cholestan-3β-yl-propionate and 5α-hydroxy-6β-[2-(1H-imidazol-4-yl)-ethylamino]-cholestan-3β-ol at 100 nM, 1 μM and 10 μM. A control (CTL) is also carried out using the protocol described above but without treatment with the aforementioned compounds. Cell survival is quantified by a trypan blue assay with automatic counting with the Biorad TC20 apparatus (TC20™ Automated Cell Counter). The trypan blue assay is based on the integrity of the cell membrane, which is broken in dead cells. The trypan blue stains the dead cells blue. The Biorad TC20 cell counter counts the proportions of blue cells and non-blue cells, relative to the cell percentages. The results are shown in FIG. 5. FIG. 5 shows, on the ordinate, the percentage cell survival relative to the control group.

It is shown in FIG. 5 that for 100 nM of treatment with the compounds 5α-hydroxy-6β-[2-(1H-imidazol-4-yl)-ethylamino]-cholestan-3β-yl propionate and 5α-hydroxy-6β-[2-(1H-imidazol-4-yl)-ethylamino]-cholestan-3β-ol, the percentage of live cells remains unchanged compared to the control group (CTL). Furthermore, for a concentration of 1 μM of 5α-hydroxy-6β-[2-(1H-imidazol-4-yl)-ethylamino]-cholestan-3β-ol, 5α-hydroxy-6β-[2-(1H-imidazol-4-yl)-ethylamino]-cholestan-3β-yl propionate, the percentage cell survival is respectively 85.42% with a standard deviation (SD) equal to 4.73 (i.e. 85.42±4.73%), and 84.08±4.09%. For a concentration of 10 μM of 5α-hydroxy-6β-[2-(1H-imidazol-4-yl)-ethylamino]-cholestan-3β-ol, 5α-hydroxy-6β-[2-(1H-imidazol-4-yl)-ethylamino]-cholestan-3β-yl propionate, the percentage cell survival is 43.25%±2.44, and 30.46±5.22%, respectively.

In conclusion, we observe cytotoxic activity of the compounds of formula (I) toward the Neuro2a tumor cells for concentrations of 1 μM for 5α-hydroxy-6β-[2-(1H-imidazol-4-yl)-ethylamino]-cholestan-3β-yl propionate and 10 μm for 5α-hydroxy-6β-[2-(1H-imidazol-4-yl)-ethylamino]-cholestan-3β-yl propionate.

### Example 20: Effect of the Compound 5α-hydroxy-6β-[2-(1H-imidazol-4-yl)-ethylamino]-cholestan-3β-yl propionate or DX107 on the Viability of MCF-7 Cells

A cell viability test was carried out on MCF-7 mammary tumor cells (Michigan Cancer Foundation-7) overexpressing HER2 (ER(+) cells) containing receptors for the hormone estrogen.

The MCF-7 cells are in a cell culture medium identical to example 14 and are seeded in 12-well plates at 50000 cells per well for 24 h. 24 hours after seeding, the cells are treated with the solvate vehicle comprising water and ethanol with an ethanol ratio 1‰ and comprising 5α-hydroxy-6β-[2-(1H-imidazol-4-yl)-ethylamino]-cholestan-3β-yl propionate or 5α-hydroxy-6β-[2-(1H-imidazol-4-yl)-ethylamino]-cholestan-3β-ol at 1, 2.5 or 5 μM. The cells are observed in an inverted microscope and photographed with the microscope camera at 24 h and 48 h. The morphological changes of the cells at 1 μM are very slight. Some white vesicles are observed after 24 hours of treatment with 5α-hydroxy-6β-[2-(1H-imidazol-4-yl)-ethylamino]-cholestan-3β-ol.

In this test:
The white vesicles reflect cell death by cytotoxic autophagy;
The cells that become rounded reflect cell death;
The cells that become detached reflect a cytotoxic effect;
The adherent cells reflect cell survival;
The supernatant cells reflect cell death; and
The refractive cells reflect cell death.

[Table 1] Morphology of the MCF7 cells after 24 hours and 48 hours of treatment with 5α-hydroxy-6β-[2-(1H-imidazol-4-yl)-ethylamino]-cholestan-3β-ol and its prodrug derivative:

| Treatment | Observations at 24 hours | Observations at 48 hours |
|---|---|---|
| 5α-hydroxy-6β-[2-(1H- | Cytostatic effet. | The cytostatic effect is more |
| imidazol-4-yl)-ethylamino]- | Numerous white vesicles. | marked. There are more |
| cholestan-3β-ol at 2.5 μM | Some cells rounded | cells that become rounded |
| (DX101) | | compared to observation at |
| | | 24 h. |
| 5α-hydroxy-6β-[2-(1H- | Almost all cells supernatant | All the cells are supernatant. |
| imidazol-4-yl)-ethylamino]- | (fairly refractive). | |
| cholestan-3β-ol at 5 μM | Occasional adherent cells | |
| (DX101) | with vesicles. | |
| 5α-hydroxy-6β-[2-(1H- | Lower cytotoxic effet than | Presence of some white |
| imidazol-4-yl)-ethylamino]- | 5α-hydroxy-6β-[2-(1H- | vesicles and rounded cells. |
| cholestan-3β-yl propionate | imidazol-4-yl)-ethylamino]- | |
| at 2.5 μM | cholestan-3β-ol. | |
| (DX107) | However, presence of some | |
| | white vesicles. | |
| 5α-hydroxy-6β-[2-(1H- | Adherent cells with white | Almost all the cells are |
| imidazol-4-yl)-ethylamino]- | vesicles. | supernatant. |
| cholestan-3β-yl propionate | The effect is similar to 5α- | Some occasional adherent |
| at 5 μM | hydroxy-6β-[2-(1H-imidazol- | cells. |
| (DX107) | 4-yl)-ethylamino]-cholestan- | |
| | 3β-ol at 2.5 μM. | |

The observations described above show that the compounds of formula (I) display cytotoxic activity on the MCF-7 cells. Based on the microscope observations, the compounds can be classified with respect to their activity, leading to cells that become rounded and white vesicles:
At 24 hours: 5α-hydroxy-6β-[2-(1H-imidazol-4-yl)-ethylamino]-cholestan-3β-ol>5α-hydroxy-6β-[2-(1H-imidazol-4-yl)-ethylamino]-cholestan-3β-yl propionate;
At 48 hours: 5α-hydroxy-6β-[2-(1H-imidazol-4-yl)-ethylamino]-cholestan-3β-ol>5α-hydroxy-6β-[2-(1H-imidazol-4-yl)-ethylamino]-cholestan-3β-yl propionate.

Following these observations, a cell viability test is measured by MTT labeling at 48 hours. This test is based on the use of the tetrazolium salt MTT (3-(4,5-dimethylthiazol-2-yl)-2,5-diphenyltetrazolium bromide). Tetrazolium is reduced by mitochondrial succinate dehydrogenase of active living cells, in formazan, precipitate of violet color. The amount of precipitate formed is proportional to the quantity of living cells but also to the metabolic activity of each cell. Thus, a simple determination of the optical density at 540 nm by spectroscopy reveals the relative quantity of living and metabolically active cells. After 48 hours, the medium is aspirated, the cells are washed with phosphate-buffered saline (PBS) and then incubated with MTT (0.5 mg/ml in PBS) for about 2 hours. The MTT solution is aspirated and then the violet crystals are dissolved in dimethylsulfoxide (DMSO). The OD (optical density) is measured at 540 nm.

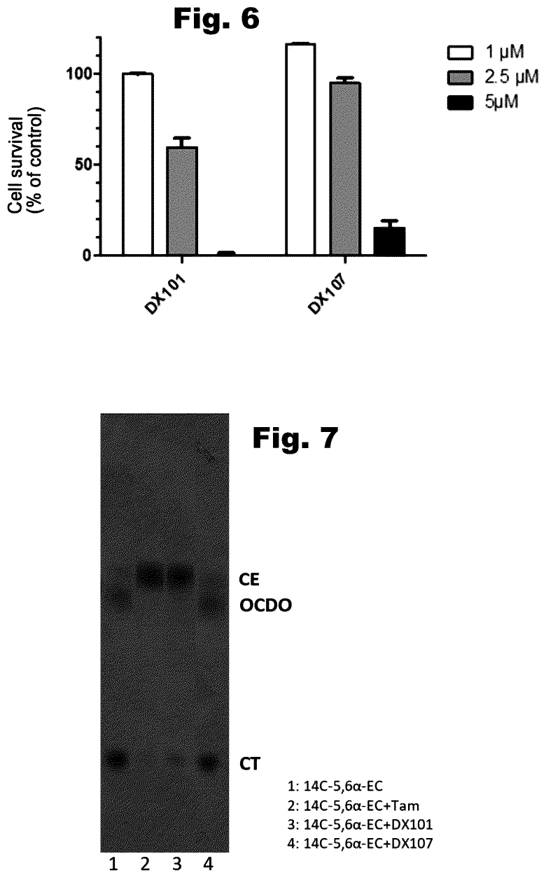

The results of this assay are presented in FIG. 6. FIG. 6 shows on the ordinate the percentage cellular viability relative to the control group. The control group is carried out similarly to the study groups without adding the molecules studied in the present text. Compared to the control, a decrease is measured, dependent on the dose of 5α-hydroxy-6β-[2-(1H-imidazol-4-yl)-ethylamino]-cholestan-3β-ethoxy, in cellular viability in MTT for 5α-hydroxy-6β-[2-(1H-imidazol-4-yl)-ethylamino]-cholestan-3β-yl propionate and 5α-hydroxy-6β-[2-(1H-imidazol-4-yl)-ethylamino]-cholestan-3β-ol.

For a concentration of 2.5 μM, a decrease in cellular viability is observed for the MCF-7 cells relative to the control group. Treatment with the compound 5α-hydroxy-6β-[2-(1H-imidazol-4-yl)-ethylamino]-cholestan-3β-ol leads to a cellular viability close to 60% relative to the control group. For the compound 5α-hydroxy-6β-[2-(1H-imidazol-4-yl)-ethylamino]-cholestan-3β-yl propionate, cellular viability is about 95%. In conclusion, for treatment with a concentration of 2.5 μM, the following order of efficacy is obtained: 5α-hydroxy-6β-[2-(1H-imidazol-4-yl)-ethylamino]-cholestan-3β-ol>5α-hydroxy-6β-[2-(1H-imidazol-4-yl)-ethylamino]-cholestan-3β-yl propionate.

For treatment at a concentration of 5 μM, a decrease is observed in cellular viability for the MCF-7 cells relative to the control group. For treatments with the compounds 5α-hydroxy-6β-[2-(1H-imidazol-4-yl)-ethylamino]-cholestan-3β-ol, 5α-hydroxy-6β-[2-(1H-imidazol-4-yl)-ethylamino]-cholestan-3β-yl propionate, cellular viability is about 18%. In conclusion, for treatment with a concentration of 5 μM, the following order of efficacy is obtained: 5α-hydroxy-6β-[2-(1H-imidazol-4-yl)-ethylamino]-cholestan-3β-ol>5α-hydroxy-6β-[2-(1H-imidazol-4-yl)-ethylamino]-cholestan-3β-yl propionate.

This experiment demonstrates a capacity for destruction of mammary tumor cells by the compounds of formula (I). These results are in agreement with the aforementioned observations made at 24 h and 48 h.

### Example 21: Effect of the Compound 5α-hydroxy-6β-[2-(1H-imidazol-4-yl)-ethylamino]-cholestan-3β-yl propionate or DX107 on the activity of cholesterol epoxide hydrolase (ChEH) in MCF-7 Cells

The compounds 5,6α-epoxycholesterol (5,6α-EC) and 5,6β-epoxycholesterol (5,6β-EC) are oxysterols involved in the anticancer pharmacology of tamoxifen, a widely used antitumor drug. They are both metabolized in cholestan-3β,5α,6β-triol (CT) by the enzyme cholesterol-5,6-epoxide hydrolase (ChEH), and CT is metabolized by the enzyme HSD11B2 (11β-hydroxysteroid dehydrogenase 2) to 6-oxo-cholestan-3β,5α-diol (OCDO), an oncosterone tumor promoter. The aim of the following experiment is to demonstrate the capacity of the compound 5α-hydroxy-6β-[2-(1H-imidazol-4-yl)-ethylamino]-cholestan-3β-yl propionate to block ChEH and therefore to limit the metabolization of oncosterone, a tumor promoter metabolite.

MCF-7 cells are in a cell culture medium identical to example 20 and are seeded in 6-well plates at 150000 cells per well with 3 wells per treatment condition. 24 h after seeding, the MCF-7 cells are treated with [¹⁴C]5,6α-EC (stock solution 1000×: 0.6 mM; 20 μCi/μmol; final concentration 0.6 μM) alone or in combination with tamoxifen (tam). Tamoxifen is used as positive control of the study compounds. The treatment with the compound according to the invention is carried out at a concentration of 1 μM.

After 24 hours of treatment, the media are collected and lipid extracts are prepared from the cell pellets by extraction with 100 μL of chloroform, 400 μL of methanol and 300 μL of water. The lipid extracts are analyzed by thin-layer chromatography (TLC) using ethyl acetate (EtOAc) as eluent. The analysis is carried out using a plate reader and then by autoradiography.

The results are presented in FIG. 7. We observe almost complete metabolization of the epoxide to CT and OCDO (well 1) and total inhibition of ChEH activity by tamoxifen and almost total (trace of CT) by 5α-hydroxy-6β-[2-(1H-imidazol-4-yl)-ethylamino]-cholestan-3β-ol. For 5α-hydroxy-6β-[2-(1H-imidazol-4-yl)-ethylamino]-cholestan-3β-yl propionate, inhibition of ChEH is observed relative to the control (1), but it is less than the positive control (2) and 5α-hydroxy-6β-[2-(1H-imidazol-4-yl)-ethylamino]-cholestan-3β-ol. Based on these results, the following order of efficacy is deduced: 5α-hydroxy-6β-[2-(1H-imidazol-4-yl)-ethylamino]-cholestan-3β-ol>5α-hydroxy-6β-[2-(1H-imidazol-4-yl)-ethylamino]-cholestan-3β-yl propionate. In conclusion, the study compounds possess inhibitory activity on ChEH. It is observed that the order of efficacy is identical to the order established for the morphological changes observed after 24 hours of treatment for the MCF-7 cells in example 20.

### Example 22: Study of the Cytotoxicity of the DX101 Prodrugs on 4T1 Cells

The cellular viability tests were carried out on murine mammary tumor 4T1 cells characterized as negative triples (HER2−, ER−, PR−).

For this experiment, a cell culture medium was prepared. The culture medium consists of Dulbecco's Modified Eagle Medium (DMEM, marketed by Westburg under the reference LO BE12-604F), comprising 4.5 g/L glucose with L-glutamine, to which 10% of fetal calf serum (FCS) and 50 U/mL of penicillin/streptomycin are added. The 4T1 cells are introduced into this culture medium.

96-well plates were seeded with 2000 4T1 cells per well. After 72 hours (h) of culture in normal conditions, i.e. in an incubator at a temperature of 37° C. at 5% CO₂, the 4T1 cells are treated for 48 h with DX101, DX107, DX113, DX117, DX119, DX121 or DX131 at 100 nM, 1 μM, 2.5 μM and 10 μM. A control condition (CTL) is also carried out in parallel using the protocol described above but without treatment with the molecules DX101, DX107, DX113, DX117, DX119, DX121, DX131 or DX133.

Cellular viability is measured by three different methods. For the first method, MTT labeling is carried out at 48 hours. This test is based on the use of the tetrazolium salt MTT (3-(4,5-dimethylthiazol-2-yl)-2,5-diphenyltetrazolium bromide). The tetrazolium is reduced by mitochondrial succinate dehydrogenase of active living cells, to formazan, a precipitate of a violet color. The quantity of precipitate formed is proportional to the quantity of living cells but also to the metabolic activity of each cell. Thus, a simple determination of the optical density at 550 nm by spectroscopy reveals the relative quantity of living and metabolically active cells. After 48 hours, the medium is aspirated, and the cells are incubated with MTT (0.5 mg/ml in culture medium) for about 3 hours. The MTT solution is aspirated and the violet crystals are dissolved in dimethylsulfoxide (DMSO). The OD (optical density) is measured at 550 nm. The percentage viability is then determined in each well relative to the CTL and the IC₅₀ (concentration for which there are still 50% of living cells) is determined for each molecule with Prism software using the nonlinear regression straight line (log(inhibitor) vs. Response).

For the second method, the percentage viability is determined using assay of the activity of the enzyme LDH (lactate dehydrogenase) in the cellular supernatants using the kit non radioactive cytotoxicity assay kit (Promega). LDH is an enzyme released in the supernatant of dead cells. The higher the LDH activity in the supernatant, the greater is the cell death. In this enzyme test, the LDH released converts a violet tetrazolium salt to formazan, of red color, absorbing at 490 nm. The intensity of the red color is proportional to the number of dead cells. After 48 h of treatment, the supernatants are transferred to a new 96-well plate and are incubated for 30 minutes in the presence of the substrate mix at room temperature. The reaction is stopped using the stop buffer and the absorbance is determined at 490 nm. The percentage cell death is determined here using a control 100% of maximum activity of LDH (carried out with untreated cells incubated in the presence of the lysis solution for 45 minutes at 37° C. just before adding the substrate mix), and the cellular viability in each well is then deduced from this percentage. IC₅₀ is then determined as explained in the preceding paragraph.

For the third method, the percentage viability is determined using the kit CellTox Green Cytotoxicity Assay (Promega). This assay measures cell death via a change in membrane integrity. This assay uses a probe of the cyanin type which does not enter the cells when they are living, but which binds to the DNA of dead cells, being permeable to the probe, making it fluorescent. As a result, the higher the fluorescence in the wells, the greater is the cell death. After 48 h of treatment, the cells are incubated for a minimum of 15 minutes in the presence of the Celltox green reagent at room temperature and the fluorescence is read at λₑₘᵢₛₛᵢₒₙ 485 nm/λ_{excitation} 590 nm. The percentage cell death is determined using the control 100% of cell death (carried out for untreated cells incubated in the presence of the lysis solution for 30 minutes at 37° C. before adding the Celltox green reagent), and the cellular viability in each well is then deduced from this percentage. IC₅₀ is then determined as explained above.

The results for IC₅₀ of these assays are presented in Tables 2a, 2b and 2c. In these tables:
^{a} the significance threshold was calculated by comparing the LogIC₅₀ of the compound against the LogIC₅₀ of DX101 with min n=3 and a one-way ANOVA test followed by a Dunnett's post-test
n^{b} represents the independent test number with 4 to 10 replicates for each condition.

TABLE 2a

| | | MTT | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 15 | 0.65 ± 0.05 | 4.47 | / |
| DX107 | 4 | 0.58 ± 0.06 | 3.79 | >0.05 |
| DX113 | 6 | 1.26 ± 0.08 | 18.20 | <0.0001 |
| DX117 | 5 | 1.04 ± 0.07 | 10.94 | <0.0001 |
| DX119 | 4 | 0.97 ± 0.03 | 9.23 | <0.01 |
| DX121 | 6 | 1.04 ± 0.05 | 10.84 | <0.001 |
| DX131 | 3 | 1.41 ± 0.03 | 25.70 | <0.0001 |
| DX133 | 1 | 1.09 ± 0.22 | 12.25 | / |

TABLE 2b

| | | LDH | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 14 | 0.53 ± 0.06 | 3.39 | / |
| DX107 | 4 | 0.52 ± 0.03 | 3.29 | >0.05 |
| DX113 | 7 | 0.72 ± 0.05 | 5.28 | >0.05 |
| DX117 | 4 | 0.92 ± 0.13 | 8.31 | <0.01 |
| DX119 | 4 | 0.49 ± 0.11 | 3.12 | >0.05 |
| DX121 | 5 | 0.67 ± 0.11 | 4.69 | >0.05 |
| DX131 | 1 | 0.83 ± 0.19 | 6.76 | / |
| DX133 | 1 | 0.62 ± 0.11 | 4.17 | / |

TABLE 2c

| | | Green Cytotoxicity Assay | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 11 | 0.55 ± 0.08 | 3.55 | / |
| DX107 | 4 | 0.37 ± 0.13 | 2.33 | >0.05 |
| DX113 | 7 | 1.04 ± 0.10 | 10.91 | <0.01 |
| DX117 | 5 | 0.98 ± 0.12 | 9.54 | <0.05 |
| DX119 | 5 | 0.56 ± 0.14 | 3.67 | >0.05 |
| DX121 | 6 | 0.82 ± 0.11 | 6.59 | <0.05 |
| DX131 | 2 | 0.23 ± 0.02 | 1.81 | / |
| DX133 | 1 | 0.55 ± 0.17 | 3.56 | / |

It is shown in Tables 2a, 2b and 2c that for the activity of DX107, IC₅₀ is equivalent to that of DX101, indicating a cytotoxic activity similar to that of DX101. Furthermore, the activities of the other molecules tested, DX113, DX117, DX119, DX121, DX131 and DX133 are lower or equivalent to that of DX101.

### Example 23: Study of the Cytotoxicity of the Prodrugs of DX101 on BT-474 Cells

The cellular viability tests were also carried out on BT-474 human mammary tumor cells (characterized as being positive triples HER2+, ER+, PR+). The BT-474 cells are in a cell culture medium identical to the preceding example and are seeded in 24-well plates at 70 000 cells per well, for determination of cellular viability using trypan blue, or in 96-well plates at 13 000 cells per well for determination of cellular viability using the MTT or LDH assay. After 96 hours (h) of culture in normal conditions, i.e. in an incubator at a temperature of 37° C. at 5% CO₂, the BT-474 cells are treated for 48 h with DX101, DX107, and DX113 at 100 nM, 1 μM, 2.5 μM and 10 μM. A control is also carried out using the protocol described above but without treatment with the molecules DX101, DX107, DX113, DX117, DX119, DX121 or DX131.

After trypsin digestion for 10 minutes at 37° C., cell survival was quantified, also by a trypan blue assay with automatic counting with the Biorad TC20 equipment (TC20™ Automated Cell Counter). The trypan blue assay is based on the cell membrane integrity, which is broken in the dead cells. Trypan blue stains the dead cells blue. The Biorad TC20 cell counter counts the proportion of blue cells and non-blue cells, relative to the cell percentages. The percentage viability is then determined in each well relative to the untreated cells and the IC₅₀ is determined as explained in the preceding example. The results are shown in Table 2. The percentage viability of the BT-474 cells was determined using the MTT and LDH assay, carried out as described in the preceding example. The results are also shown in Table 2.

The results for IC₅₀ from these assays are presented in Tables 3a, 3b and 3c. In these tables:
^{a} The significance threshold was calculated by comparing LogIC₅₀ of the compound against LogIC₅₀ of DX101 with min n=3 and a one-way ANOVA test followed by a Dunnett's post-test
n^{b} represents the independent test number with 4 to 10 replicates for each condition.

TABLE 3a

| | | MTT | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 11 | 0.85 ± 0.02 | 7.06 | / |
| DX107 | 6 | 1.08 ± 0.04 | 11.89 | <0.05 |
| DX113 | 7 | 1.33 ± 0.07 | 21.43 | <0.0001 |
| DX117 | 1 | 0.79 ± 0.06 | 6.17 | / |
| DX119 | 2 | 1.33 ± 0.06 | 14.45 | / |
| DX121 | 1 | 1.05 ± 0.17 | 11.22 | / |
| DX131 | 1 | 1.08 ± 0.08 | 12.02 | / |

TABLE 3b

| | | LDH | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 7 | 0.99 ± 0.06 | 9.72 | / |
| DX107 | 3 | 0.95 ± 0.08 | 8.92 | >0.05 |
| DX113 | 6 | 1.38 ± 0.07 | 23.71 | <0.01 |
| DX117 | 1 | 0.72 ± 0.18 | 5.25 | / |
| DX119 | 2 | 1.41 ± 0.01 | 25.41 | / |
| DX121 | 1 | 1.47 ± 0.40 | 11.75 | / |
| DX131 | 1 | 1.45 ± 0.08 | 28.18 | / |

TABLE 3c

| | | Trypan blue | | |
|---|---|---|---|---|
| IC₅₀ (μM) | | Mean LogIC₅₀ | IC₅₀ | |
| Compound | n^{b} | (±SEM) | (μM) | p value^{a} |
| DX101101 | 6 | 0.61 ± 0.20 | 4.03 | / |
| DX107 | 6 | 0.66 ± 0.04 | 4.58 | >0.05 |
| DX113 | 6 | 1.09 ± 0.15 | 12.30 | >0.05 |

It is shown in Tables 3a, 3b and 3c that the activity of DX107 is similar to that of DX101, and that the activity of DX1 13 is lower than that of DX101 in this line. The activity of DX1 17 seems to be equivalent to that of DX101 and the activities of the other compounds tested, DX119, DX121 and DX131 seem to be lower than that of DX101.

### Example 24: Effect of the Prodrug DX107 on Tumor Growth In Vivo

All the procedures on the animals were carried out in accordance with the guidelines of our institution after being approved by the ethics committee. The 4T1 cells were cultured as before, and were dissociated in trypsin and washed twice in cold PBS and resuspended in PBS at 1.5 million/mL. The 4T1 tumors were obtained by subcutaneous transplantation of 0.150 million cells in 100 μL in the flank of female Balb/c mice (9 weeks, January). When the tumors reached a volume of 50-100 mm³, the mice were fed by stomach tube with 40 mg/kg of DX101 or 40 mg/kg of DX111 or the control vehicle (water). The treatment was carried out every day until the end of the experiment (tumor volume >1000 mm³). The tumor volume was determined every day using a caliper gauge and calculated from the formula: ½×(Length*Width²). The percentage inhibition of tumor growth was determined from the following formula: 100×(1−(tumor volume, day 8/tumor volume day 0)_{DX107})/(1−(tumor volume, day 8/tumor volume day 0)_{vehicle}).

The Kaplan-Meier method was used for comparing the animals' survival.

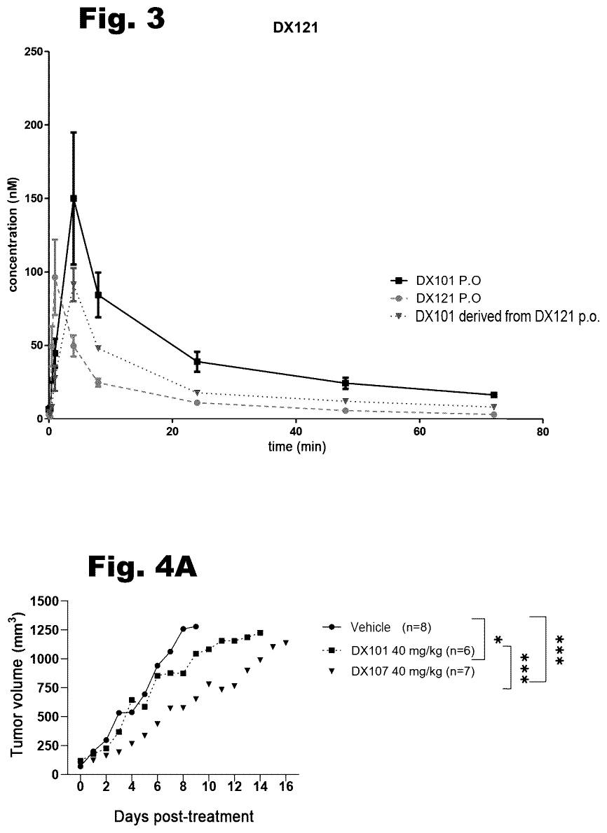

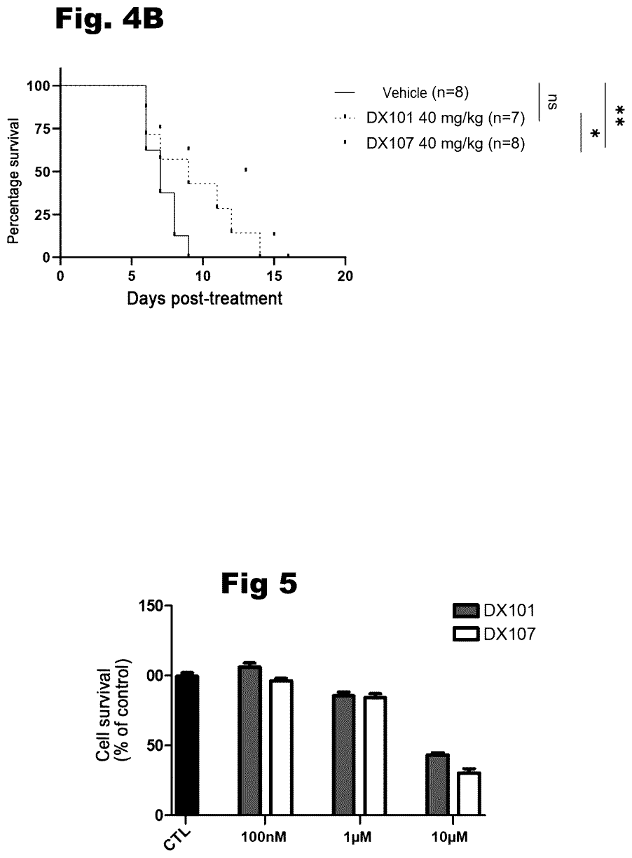

FIG. 4A shows that DX107 has a greater effect than DX101 on reduction of tumor growth (***p<0.001, one-way ANOVA test and Tukey post-test). Inhibition of tumor growth was in addition determined at 78% for the animals treated with DX107 and at 58% for the animals treated with DX101.

Furthermore, analysis of the animals' survival, also shown in FIG. 4B, indicates a median survival significantly higher for the animals treated with DX107 compared to that of DX101 (Log-rank Mantal-Cox test, *p<0.05).

In FIG. 4B, the results obtained are as follows:

Median survival (days)

| Vehicle | DX101 (40 mg/kg) | DX107 (40 mg/kg) |
|---|---|---|
| 7 | 9 | 14 |

Although the invention has been described in connection with several particular embodiments, it is quite clear that it is not in any way limited to these, and that it comprises all the technical equivalents of the means described as well as their combinations if the latter fall within the scope of the invention.

The use of the verb “comprise” or “include” and its conjugated forms does not exclude the presence of other elements or of other steps than those stated in a claim.

In the claims, any reference symbol in parentheses is not to be interpreted as a limitation of the claim.

1. A compound of formula (I); 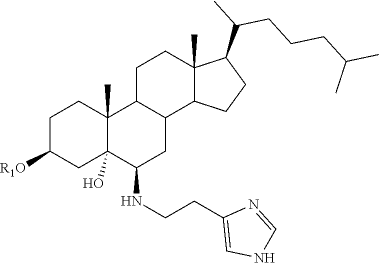
or a pharmaceutically acceptable salt thereof, in which R₁ is selected from:
a group —C(O)NR₂R₃, where R₂, R₃ are equivalent or different and are selected from H and a linear, saturated C1 to C8 carbon chain containing the substituent 1-H-imidazol-4-yl,
a group —C(O)R₄, where R₄ is the radical —CH₂CH₃,
a group —C(O)OR₅, where R₅ is a C1 to C8 carbon chain,
a group —C(O)CHNH(COCH₂CH₃)R₆ where R₆ is the side chain of the amino acids selected from —CH₂—C₃N₂H₂, —CH₂CH(CH₃)₂, —CH(CH₃)CH₂CH₃, —CH(CH₃)₂, —CH₂C₆H₅, —CH₂C₈NH₆, —(CH₂)₄NH₂, —CH₂C₆OH₅, —C₃H₅N, for use as a drug for causing regression of a mammalian cancerous tumor.

2. The compound as claimed in claim 1, in which R₁ is a group —C(O)R₄ where R₄ is the radical —CH₂CH₃.

3. The compound as claimed in claim 1, in which R₁ is a group —C(O)OR₅, where R₅ is an ethyl or butyl carbon chain.

4. The compound as claimed in claim 1, in which R₁ is a group —C(O)NR₂R₃ where R₂, R₃ are equivalent or different and are selected from H and a linear, saturated C1 to C8 carbon chain containing the substituent 1-H-imidazol-4-yl.

5. The compound as claimed in claim 1, in which R₁ is a group —C(O)CHNH(COCH₂CH₃)R₆ where R₆ is the side chain of the amino acids selected from CH₂—C₃N₂H₂, CH₂CH(CH₃)₂, CH(CH₃)CH₂CH₃, CH(CH₃)₂, CH₂C₆H₅, CH₂C₈NH₆, (CH₂)₄NH₂, CH₂C₆OH₅, C₃H₅N.

6. The compound as claimed in claim 1 in which the compound of formula (I) is selected from:
5α-hydroxy-6β-[2-(1H-imidazol-4-yl)-ethylamino]-cholestan-3β-propionate;
5α-hydroxy-6β-[2-(1H-imidazol-4-yl)-ethylamino]-cholestan-3β-hexanoate;
5α-hydroxy-6β-[2-(1H-imidazol-4-yl)-ethylamino]-cholestan-3β-yl-ethyl carbonate;
5α-hydroxy-6β-[2-(1H-imidazol-4-yl)-ethylamino]-cholestan-3β-yl butyl carbonate;
5α-hydroxy-6β-[2-(1H-imidazol-4-yl)-ethylamino]-cholestan-3β-yl 1-H-imidazol-4-yl ethyl carbamate;
5α-hydroxy-6β-[2-(1H-imidazol-4-yl)-ethylamino]-cholestan-3β-yl-ethyl carbamate;
L-histidine 5α-hydroxy-6β-[2-(1H-imidazol-4-yl)-ethylamino]-cholestan-3β-yl ester,
L-leucine 5α-hydroxy-6β-[2-(1H-imidazol-4-yl)-ethylamino]-cholestan-3β-yl ester,
L-isoleucine 5α-hydroxy-6β-[2-(1H-imidazol-4-yl)-ethylamino]-cholestan-3β-yl ester,
L-valine 5α-hydroxy-6β-[2-(1H-imidazol-4-yl)-ethylamino]-cholestan-3β-yl ester,
L-phenylalanine 5α-hydroxy-6β-[2-(1H-imidazol-4-yl)-ethylamino]-cholestan-3β-yl ester,
L-tryptophan 5α-hydroxy-6β-[2-(1H-imidazol-4-yl)-ethylamino]-cholestan-3β-yl ester,
L-lysine 5α-hydroxy-6β-[2-(1H-imidazol-4-yl)-ethylamino]-cholestan-3β-yl ester,
L-tyrosine 5α-hydroxy-6β-[2-(1H-imidazol-4-yl)-ethylamino]-cholestan-3β-yl ester,
L-proline 5α-hydroxy-6β-[2-(1H-imidazol-4-yl)-ethylamino]-cholestan-3β-yl ester.

7. The compound as claimed in claim 1, in which the cancerous tumor is a chemosensitive cancer.

8. The compound as claimed in claim 1, in which the cancerous tumor is a drug-resistant cancer.

9. The compound as claimed in claim 8, in which the drug-resistant cancer is a hematologic or blood cancer, such as leukemia, in particular acute myeloid leukemia or acute lymphocytic leukemia, lymphoma, in particular non-Hodgkin lymphoma and multiple myeloma.

10. The compound as claimed in claim 8, in which the cancer is drug-resistant to daunorubicin, cytarabine, fluorouracil, cisplatin, all-trans retinoic acid, arsenic trioxide, bortezomib or a combination thereof.

11. A pharmaceutical composition comprising, in a pharmaceutically acceptable vehicle, at least one compound as claimed in claim 1 for use as a drug for causing regression of a mammalian cancerous tumor.

12. The pharmaceutical composition as claimed in claim 11, comprising at least one other therapeutic agent.

13. The pharmaceutical composition as claimed in claim 12, in which the other therapeutic agent is an antineoplastic agent.

14. The pharmaceutical composition as claimed in claim 13 for use in cancer treatment in a patient with a tumor that is drug-resistant to the antineoplastic agent when it is not administered as part of the pharmaceutical composition.

15. The pharmaceutical composition as claimed in claim 14 for use in cancer treatment in a patient with a tumor that is chemosensitive to the antineoplastic agent, and the dose of the antineoplastic agent administered to the patient as part of the pharmaceutical composition or a pharmaceutically acceptable salt thereof is lower than the dose of the antineoplastic agent when it is not administered as part of the pharmaceutical composition.

16. The pharmaceutical composition as claimed in claim 11, wherein it is in a suitable form to be administered intravenously, subcutaneously, intraperitoneally, or orally.

17. A method of treating cancer in a patient comprising the step of administering a therapeutically effective dose of the pharmaceutical composition as claimed in claim 11.
